(19) United States
(12) Patent Application Publication
(10) Pub. No.: US 2004/0029159 A1
(43) Pub. Date: Feb. 12, 2004

(54) Nucleic acid switch patterns as cell or tissue type identifiers

(76) Inventors: William J. Dreyer, Pasadena, CA (US); Janet Roman-Dreyer, Pasadena, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/440,493

(22) Filed: May 15, 2003

Related U.S. Application Data
(63) Continuation-in-part of application No. 09/887,551, filed on Jun. 22, 2001, now abandoned.
(63) Continuation-in-part of application No. 09/366,458, filed on Aug. 3, 1999.
(60) Provisional application No. 60/213,620, filed on Jun. 23, 2000.
(60) Provisional application No. 60/095,148, filed on Aug. 3, 1998.

(51) Int. Cl. C12Q 1/68

(52) U.S. Cl. 435/6

(57) ABSTRACT

Methods are provided for characterizing a developmental or lineage-specific cell type or other cell types by analyzing nucleic acid switch patterns or profiles and/or proteins indicative of these switches. Such a method can include, for example, comparing the nucleic acid of the cell with nucleic acid from a corresponding germline cell or other cell, wherein a difference in the nucleic acid is indicative of a nucleic acid switch. Optionally, the cell type can be further characterized in terms of developmental or lineage specific cell type.

### CROSS REFERENCE TO RELATED APPLICATION(S)

This application is a Continuation-in-Part of U.S. Ser. No. 09/887,551, filed Jun. 22, 2001, which claims priority under 35 U.S.C. 119(e) to U.S. Ser. No. 60/213,620, filed Jun. 23, 2000, and is a Continuation-in-Part of U.S. Ser. No. 09/366,458, filed Aug. 3, 1999, which claims priority under 35 U.S.C. 119(e) to U.S. Ser. No. 60/095,148, filed Aug. 3, 1998, the entire content of each of which is incorporated herein by reference.

### BACKGROUND OF THE INVENTION

### FIELD OF THE INVENTION

The present invention relates generally to cell lineage determination and more particularly to genetic switches and mobile element related genes and their role in genetic programming during development or cell lineage decisions

### BACKGROUND INFORMATION

There is a need for precise genetic programming of development. Consider the fascinating phenomenon of identical twins. Each twin is not simply similar to his or her sibling, but shares every physical attribute that can be perceived, including aspects of brain structure, behavioral mannerisms and parallel changes with aging. Consider also a spider, with the ability to form its own species-determined web architecture. And consider the reproducible color patterns of butterfly wings and tropical fish. How is such developmental precision achieved? Currently accepted theories of development invoking epigenetic mechanisms do not fully address the question of how a DNA program can generate identical developmental outcomes with such remarkable reproducibility in separate individuals.

The developing immune system, which utilizes programmed genetic switching as distinct cell lineages are formed, has long seemed to us not an aberrant phenomenon but an instructive model for studying other developing systems. The programmed DNA alterations occurring during development of B cells and T cells are an example of a genetic mechanism that achieves the precision, control and cell lineage memory lacking in epigenetic theories of development. Recently, evidence has been collected indicating that DNA switching does, in fact, occur outside of the immune system, in particular in the control element sequences of the olfactory receptors, a class of receptors found in numerous tissues other than the olfactory system.

The “Area Code Hypothesis” helps explain how chromosomes sculpture living organisms. The DNA contained in the two cells that will form identical twins is able to choreograph the parallel development of two strikingly similar individuals through birth and through all of the stages of their lives. In a favorable environment the twins will grow, rearrange their bodies at puberty, and go through the changes of maturity and aging in parallel. Even the MRI images of their brains will be strikingly similar and very different from other brain images. It was consideration of this extraordinary precision of cell and neural assembly that originally lead to the proposition of the Area Code Hypothesis (1; references cited by “numbers” herein are listed following Example 3). The hypothesis was based on extensive genetic, molecular, and cellular studies of the immune system (2,3; see also refs. in (1)).

Key elements of the hypothesis are the following: 1) Large multigene families must exist that code for cell surface receptors providing highly specific cell-cell recognition functions; 2) Receptors must be used repeatedly in a combinatorial fashion so that a finite number of genes can provide enough information to generate the required large number of cellular addresses; 3) Programmed genetic switching similar in some respects to that seen during the development of the immune system is assumed to aid in the complex control of the expression of these address codes in specific lineages and cells (4); and 4) Some classes of cell surface receptors are assumed to be widely expressed throughout the organism and code for large regions resembling, for example, the country codes of our telephone dialing system. Other classes of molecules would be more restricted in expression and are expected to code for multiple smaller regions of the embryo somewhat comparable, according to this metaphor, to the multiple regions specified by area codes and regional prefixes throughout the world. Finally, it is assumed that molecules exist that encode a specific cellular address comparable to the four digits used to code for a single, specific telephone in any one of the numerous, distinct topological regions specified by the earlier codes. Both the telephone digits and the genes and cell surface receptors that provide this last part of the code may be used repeatedly in diverse physical locations.

DNA switch mechanisms, such as those which occur in the immune system and which may be occurring in the olfactory gene family, are the type of genetic programming that seems necessary during development. Therefore, there is a need in the art for new and better methods for detecting DNA switch mechanisms and for treating diseases related to such DNA switch mechanisms. In addition, there is a need in the art for new and better methods for obtaining specific cell lines identified by genetic switches and/or expression of mobile element-related polynucleotides, envelope proteins and other polypeptides.

### SUMMARY OF THE INVENTION

The present invention provides methods for characterizing a developmental or lineage-specific cell type or other cell types by analyzing nucleic acid switch patterns or profiles and/or proteins indicative of these switches, wherein the nucleic acids analyzed are not nucleic acid molecules (e.g., genes) encoding immunoglobulin or T cell receptor family members and the proteins are not immunoglobulins or T cell receptor family members. The method includes comparing the nucleic acid of the cell with nucleic acid from a corresponding germline cell or other cell, wherein a difference in the nucleic acid is indicative of a nucleic acid switch. Optionally, the cell type can be further characterized in terms of developmental or lineage specific cell type. The method also includes comparing the cellular proteins with proteins from a corresponding germline cell or other cell, wherein a difference in the proteins is indicative of a nucleic acid switch; and characterizing the cell in terms of developmental or lineage specific cell type. (see Dreyer and Dreyer, Genetica 107:249-259, 1999, herein incorporated by reference in its entirety).

In another embodiment, the invention provides a method for identifying a differentiation stage-specific cell type in a cell sample. The method includes comparing nucleic acid obtained from the cells with corresponding germline or other cell nucleic acid, wherein the presence of at least one gene switch in the nucleic acid in the sample is indicative of a differentiated cell in the sample. The method also includes comparing cellular proteins with cell proteins from a corresponding germline or other cell, wherein the presence of specific proteins in the sample is indicative of a differentiated cell in the sample.

In yet another embodiment, the invention provides a method for identifying a stem cell or a stage in the stem cell lineage in a sample. The method includes contacting nucleic acid obtained from cells in the cell sample with at least one binding agent specific for a particular lineage switch such that the binding agent binds specifically to the region of nucleic acid affected by a gene switch; and detecting binding of the agent to a region of nucleic acid affected by the switch, wherein a particular switch is indicative of a stem cell stage. The method also includes contacting cellular proteins with at least one binding agent specific for a particular lineage switch such that the binding agent binds specifically to the region of the protein affected by a gene switch; and detecting binding of the agent to a region of the protein affected by the switch, wherein a particular switch is indicative of a stem cell stage

In yet another embodiment, the invention provides a method for identifying a cell in a cell sample indicative of a disease state or disease process. The method includes contacting nucleic acid from a cell suspected of having a disease with at least one binding agent specific for a nucleic acid switch such that the binding agent binds specifically to the nucleic acid or to a region of the nucleic acid indicative of a switch, wherein the specific binding of the binding agent indicates the presence of a region of nucleic acid affected by a switch, and wherein the presence of the particular switch is associated with a disease state or a disease process in the cell. The method also includes contacting proteins from a cell suspected of having a disease with at least one binding agent specific for the protein or the region of the protein resulting from a nucleic acid switch such that the binding agent binds specifically to the protein or to a region of the protein indicative of a switch, wherein the specific binding of the binding agent indicates the presence of a nucleic acid switch, and wherein the presence of the particular switch is associated with a disease state or a disease process in the cell.

In a further embodiment, the invention provides a method for diagnosing a subject having a disease or condition, at risk of having a disease, or simply having the presence of a particular nucleic acid switch which is indicative of a characteristic of the subject (e.g., predisposed to dyslexia). The method includes contacting test nucleic acid obtained from a sample of cells of the subject with at least one binding agent specific for a nucleic acid switch associated with a specific disease such that the binding agent detects a region of nucleic acid affected by the switch, wherein the binding of the agent indicates the presence or predisposition of the specific disease in the subject. The method also includes contacting cellular proteins from a sample of cells of the subject with at least one binding agent specific for proteins or regions of proteins resulting from a nucleic acid switch associated with a specific disease such that the binding agent detects a region of protein affected by the switch, wherein the binding of the agent indicates the presence of the specific disease or predisposition to a disease or condition in the subject.

In yet another embodiment, the invention provides a method for obtaining a composition substantially enriched in a specific cell type. The method includes contacting a sample of cells with at least one binding agent specific for a polynucleotide indicative of a cell type-specific nucleic acid switch such that the binding agent binds specifically to a cell or cells in the sample that binds to the polynucleotide; and separating the cell or cells bound by the binding agent from the sample, thereby obtaining a composition substantially enriched in the specific cell type. The method also includes contacting a sample of cells with at least one binding agent specific for a polypeptide indicative of a cell type-specific nucleic acid switch such that the binding agent binds specifically to a cell or cells in the sample that express the polypeptide; and separating the cell or cells bound by the binding agent from the sample, thereby obtaining a composition substantially enriched in the specific cell type.

The invention also provides a method for producing a specific cell lineage or organ type or organism. The method includes obtaining a stem cell within the cell lineage by cloning a cell identified by any of the methods of the invention as described above and treating the cell under conditions and for a time sufficient to produce the specific cell lineage, organ or organism.

The invention includes a method of obtaining a composition substantially enriched in a specific cell type. The method includes contacting a sample of cells with at least one binding agent specific for an envelope cell surface marker such that the binding agent binds specifically to a cell or cells having the marker in the sample; and separating the cell or cells bound by the binding agent from the sample, thereby obtaining a composition substantially enriched in a specific cell type.

### BRIEF DESCRIPTION OF THE DRAWINGS

FIG. 1 provides a hypothetical mechanism for the assembly of the precise topological map of glomeruli: A gradient of molecular affinities of olfactory receptors. Approximately 1,000 molecularly distinct glomeruli are arranged in a topologically precise map in the olfactory bulb, of a mouse or rat. This map is bilaterally symmetrical, but only one side is illustrated here. There are four distinct zones of glomeruli in the bulb, illustrated here in various shades of gray. Gradients of grays on glomeruli within each zone are used to suggest an orderly gradient of molecular affinities of the individual receptors. A stream of migrating neurons originates in a specific fate-mapped region of the subventricular zone. Cells migrate as streams with the growth cones of each contacting the cell ahead. Shades and gradients are used again to suggest that receptors on each cell differ in an orderly way so that neighboring cells have receptors that bind with the highest affinity to each other. After reaching the olfactory bulb, cells change their direction of migration and move toward the surface of the bulb where they generate periglomerular cells. The dendrites of these cells then form the targets for incoming growth cones of olfactory nerve axons. Hundreds of olfactory neurons bearing the same, specific, olfactory receptor converge on a single pair of bilaterally symmetrical glomeruli. Their growth cones synapse with the dendrites of the periglomerular cells presumed to express the identical receptor. These homophilic interactions occur with a higher affinity than in their heterophilic interactions. According to this hypothesis, receptors on neighboring glomeruli have closely related but different structures hence are bound with a slightly lower affinity. This provides an intriguing possible explanation for the molecular basis of the observation that olfactory axons and growth-cones bearing the identical olfactory receptor fasciculate with themselves and not their neighbors. This is illustrated by the fascicles of two different shades of gray, each seeking a different target.

FIG. 2 provide a diagram of a region of human chromosome 17 that codes for two olfactory receptors. This figure, based on the work of Glusman et al. (46), illustrates one of many sequenced regions of chromosomes that code for olfactory receptors and also contain numerous mobile elements. Note the pattern of elements near the upstream control elements of the two olfactory receptor coding regions (0R228 and OR 40; see the original publication for more details of this work. Some of these elements are hypothesized to be used as genetic switches for the control of the expression of the thousand or more olfactory receptors.

### DETAILED DESCRIPTION OF THE INVENTION

The present invention provides methods for characterizing a developmental or lineage-specific cell type by analyzing nucleic acid switch patterns or profiles in the cell and patterns of proteins indicative of such switches. The methods of the invention are based on the seminal discovery that cell surface displays of seven-transmembrane (olfactory) receptors, protocadherins and other cell surface receptors provide codes that enable cells to find their correct partners as they sculpture embryos, and that the genetic mechanisms that program the expression of such displays is achieved in part by permanent and heritable changes in DNA. Using the developing immune system as a model, two different types of developmentally programmed genetic switches, each of which relies on recombination mechanisms related to mobile elements, were examined. It should be recognized that, while the immune system is useful herein as a model for the switch patterns disclosed herein as indicative of a developmental-specific or lineage-specific cell type, the present invention does not encompass the previously described and well known immunoglobulin or T cell receptor gene switching. While not wanting to be held to a particular theory, it is believed that the involvement of mobile element related switch mechanisms is critical for cell lineage determination and development. Since both recombinase and reverse transcriptase mechanisms play a role in the switching of the immunoglobulin genes, the databases of Expressed Sequence Tags (dbEST) were searched for expression of related genes in other tissues. The present invention shows that transposases and reverse transcriptases are widely expressed in most tissues. This result strongly indicates that switch mechanisms utilizing these enzymes play a role in normal development and cell lineage determination.

Further, searches of the databases for expression of env (envelope) gene products which are cell surface molecules sometimes associated with mobile elements, were stimulated by provocative results suggesting that these molecules might function as cellular address receptors. These searches showed that env genes are also expressed in large numbers in normal human tissues. One must assume that these three different types of mobile-element-related messenger RNA molecules (transposases, reverse transcriptases, and env proteins) are expressed for use in functions of value in the various tissues, and have been preserved in the genome because of their selective advantages.

The present invention provides methods of use based on the findings that many specific cell lineage decisions are made and remembered by means of genetic switches similar to those that control the immunoglobulin and protocadherin and, probably, the seven-transmembrane/olfactory gene families and also that complex genetic programs utilizing mobile-element-related genes, program these events.

The complexity of the genetic problem of cell lineage determination and lineage memory during development can be seen using the immune system as a model. In the immune system, sophisticated alterations are made in the germline DNA as specific B or T cells are generated. Only a single allele is expressed in each cell. The altered DNA sequences are replicated for the life of a stem cell, thus accounting for the lineage memory. Genetic switching therefore remains an attractive aspect of the area code hypothesis and cell lineage determination and memory during development. Indeed, it is extremely difficult to imagine that a mechanism utilizing only transcription factors, etc., is capable of mimicking the immune system's expression of a single allele and stem cell specific receptor expression.

In zebra fish, the rag 1 recombinase is expressed in the olfactory epithelium as well as in tissues in which common and variable genes are switched in the immune system, thus adding further support to the notion of wider use of these mobile element-related mechanisms in development (Jessen et al. 1999). As disclosed herein, support is provided that recombinases and reverse transcriptases switch genes in families other than those of the immune system. The mechanism by which DNA is excised during the development of the immune system utilizes mechanisms and enzymes that evolved with mobile elements, such as DNA transposable elements and retroelements. The rag 1 transposase is evolutionarily related to the enzymes responsible for transposable element rearrangements found in essentially all eukaryotes and even to bacterial switches such as the invertrons (Spanopoulou et al. 1996; Landy 1999). Ten to twenty percent (or more) of the DNA of most multicellular organisms is made up of elements related to mobile DNA, which are referred to herein as “mobile element-related genes.” For example, large numbers of genes coding for members of the transposase/recombinase family are found in these genomes and some of these, according to the present invention, function in normal development.

During heavy chain switching in the immune system via reverse transcriptases and the related nucleases, an RNA transcript seems to function in a manner strikingly reminiscent of mechanisms used by retroelements (Muller et al. 1998). Experimental results suggest that a site-specific nuclease nicks DNA in a region of repeats termed a splice region. The RNA then forms a heterodimer with DNA in the region that was nicked. Then a reverse transcriptase copies the RNA. The net result of this process is the excision of circular DNA and the joining of the edited DNA to form a new protein coding sequence (exons and introns), control regions, etc., B cell specific retroelements are expressed in these cells and can provide the required reverse transcriptase and nuclease activities.

In general, retroelements are polynucleotide mobile elements that can exist as DNA or RNA or DNA/RNA duplexes. Although retroviruses are well known retroelements, there are many other types, including close relatives of retroviruses like LTR retrotransposons, more distant relatives like non-LTR retrotransposons, caulimoviruses and hepadnaviruses, and elements with virtually no similarity, like retrons. In the past, virtually all retroelements have been considered to be “selfish DNAs” with no involvement with the normal development or maintenance of their host cells, the only known exception being telomeres/telomerases, which maintain the ends of chromosomes (A. J. Flavell, Comp Biochem Physiol B Biochem Mol Biol 110:13-15, 1995).

The list of confirmed examples of programmed alterations in DNA is now so long that one is quite safe in stating that not all of the repeats and elements that make up a significant part of all chromosomes are “junk DNA.” In fact, examination of the cellular and molecular mechanisms associated with transposon-related elements suggests that such elements play a role in programming the expression of numerous genes, including the olfactory receptors and the protocadherins. No mechanism that does not involve alteration of DNA seems adequate to accomplish the extraordinarily complex programming of gene expression and commitment of cell lineages that is observed in both the olfactory receptor and protocadherin gene families.

Clearly, if gene switching plays a central role in lineage decisions, messenger RNA and the required enzymes for the switching machinery must be expressed in numerous tissues. The present invention is based on the seminal discovery by search of the databases of Expressed Sequence Tags (dbEST) that switch-machinery-related genes are expressed in virtually all human tissues. Because both recombinase and reverse transcriptase mechanisms play a role in the switching of the immunoglobulin genes, the search focused upon expression of related genes in other tissues.(i.e., recombinases, reverse transcriptases, and env/envelope genes). Envelope genes were included in the search for mobile element-related polypeptides because studies aimed at identifying mobile element-related polypeptides that differed on otherwise similar cell lines showed a difference in env gene products (Roman et al. 198 1). Hence, it was assumed that these mobile element-related polypeptides might also play a role in cellular addressing. Table 1 below summarizes the results of these searches.

TABLE 1

| | Recombinase | | |
|---|---|---|---|
| | (transposase/ | Reverse | Envelope |
| | integrase) | transcriptase | (env/gp70) |
| Search string | (Integrase OR | “Reverse | (Gp70 OR env |
| entered in: | transposase OR | transcriptase ” | OR envelope) |
| “Enter Search | recombinase) | AND (sapiens OR | AND (sapiens OR |
| with text . . . ” | AND (sapiens OR | human) NOT | human) NOT |
| | human) NOT | (Brugla OR mus) | (mouse OR mus) |
| | (mouse OR mus) | | |
| Number | Many hundreds | Many thousands | Many thousands |
| of human | | | |
| expressed | | | |
| sequence tags | | | |
| (ESTs) found | | | |

As can be seen by the unexpected results shown Table 1, very large numbers of recombinase, reverse transcriptase and env genes were found. Other searches revealed that these genes are also expressed in virtually every human tissue or tumor examined. The present invention is based upon the finding that expression of such mobile element-related genes takes place in a controlled, tissue and cell specific manner and that such switch machinery and mobile element-related genes play a far more important role in development than anyone has imagined. Specifically, the patterns of recombinase and reverse transcriptase expressed and functional in the developing immune system are believed to be only one manifestation of a widespread developmental mechanism involving DNA switches as cell lineages are formed. One of the consequences of cell lineage switching is the generation of combinations of polypeptides in the cell surface displays that cells use to find their correct addresses as they assemble embryos. It is believed, for example, that such combinations and patterns of expressed polypeptides function in cells as address codes.

This evidence now indicates that precise developmental control is achieved in part by permanent and heritable changes in DNA, and that machinery related to mobile elements can be involved in DNA switching that results in permanent and heritable changes in the DNA of a specific cell line. It is further believed that molecules related to mobile elements, for example envelope gene products can, therefore, be identifying characteristics of specific cell lines.

There are a number of other studies that show remarkable tissue specificity in the expression of such mobile element-related molecules. In both mice and humans, numerous retro-elements are individually expressed in a tissue-specific way, each under the control of factors appropriate for the tissue in which it is expressed. For example, epithelial growth factor can stimulate the expression of a retroelement with the appropriate target sequence in its long terminal repeat (LTR). Corticosteroids stimulate the expression of different retroelements in the adrenal glands. In addition, the LTR control sequences differ appropriately in a number of different tissues where other growth factors and hormones stimulate the expression of specific retroelements (Bohm et al. 1993; French and Norton, 1997; Medstrand and Blomberg, 1993). Evolutionary pressures could explain these results if it is assumed that these mobile elements provide a useful function when they are expressed in such a controlled and tissue-specific way.

Developmentally timed expression of env and other endogenous retroviral products have been noted with great interest (Mietz et al. 1992; French and Norton 1997; Larsson and Andersson 1998; Andersson et al. 1998; Blond et al. 1999; Lin et al. 1999). For example, the discovery of the expression of env gene products on mouse and human unfertilized oocytes, and the diminution of this expression after fertilization, raises the intriguing possibility that these gene products are involved in sperm-egg binding and fertilization (Nilsson et al. 1999).

Another remarkable study has examined the expression of more than fifteen mobile element-related genes in Drosophila tissues (Ding and Lipshitz 1994). In this study, in situ hybridization revealed RNA expression patterns that differed dramatically for almost all of the mobile element-related polypeptides and polynucleotides. The patterns are complex and definitive, reminiscent of the patterns of homeobox gene expression. In fact, the patterns of mobile-element related RNA expression evolve in time and space in a reproducible manner as embryonic development proceeds. It is believed that this extreme control evolved to serve a function.

There are numerous examples of critical functions that are performed in diverse organisms by mobile-element genes. The ciliates use recombinases to radically process the DNA of the germline micronucleus as the somatic macronucleus is created. The nematode Ascaris uses similar programmed expression of transposases, etc., to convert the germline chromosomes to radically different somatic chromosomes (Goday and Pimpinelli 1993). Drosophila uses two non-LTR retrotransposons (HeT-A and TART) to maintain telomeres (Pardue et al. 1997). Reviews of this subject that provide many additional examples of useful and programmed functions of mobile-element-related genes in organisms (e.g., Patrusky 1981; Bostock 1984; Williams et al. 1993; Medstrand and Blomberg 1993; Goto et al. 1998). An entire issue of “Trends in Genetics” was devoted to this topic (Plasterk 1992). It is believed that the mobile element-related genes found in the searches of the EST databases as disclosed herein also perform important functions in DNA processing and cell addressing; however, there can be no doubt that uncontrolled transposition of some elements also occurs. These are not mutually exclusive processes. Indeed the mobility, combined with important cellular and developmental functions, provides an important insight into mechanisms of evolution.

Perhaps the most compelling argument in favor of the role of mobile-element related mechanisms in normal development is the deleterious effects of their absence. Table 2 below provides examples of mutations in proteins that control gene rearrangements in the immune system wherein the mutations have profound effects on multiple additional tissues.

TABLE 2
Mutations in molecular mechanisms required for gene rearrangements
in the immune system result in abnormalities in other organ systems

| | | | Non-Immune-System | |
|---|---|---|---|---|
| Genetic Defect | Ig System Defect | Molecular Defect | Defects | References |
| ATM (Ataxia | Deficiency in | Inactivation of | Severe cerebellar disruption | Sedgwick and Boder |
| telangiectasia) | double-stranded | ATM protein | and wide-spread changes in | 1991; Laven and |
| | DNA joining | | the CNS Growth retardation | Shiloh 1997 |
| | | | and other developmental | |
| | | | defects | |
| NBS (Nijmegen | Deficiency in | Inactivation of | Very small brain | Featherstone and |
| Breakage | double-stranded | Nbs 1 protein | (microcephally)—50% | Jackson 1998; Paull |
| Syndrome) | DNA joining | | having low to normal | and Gellert 1999 |
| | | | intelligence Many | |
| | | | developmental defects: | |
| | | | short stature, facial bone | |
| | | | abnormalities | |
| Knockouts of | Deficiency in | Inactivation of | Embryonic lethal— | Gao et al. 1998; |
| DNA ligase IV | double-stranded | DNA ligase IV or | Neuronal precursors die | Chun and Schatz, |
| or XRCC4 | DNA joining | XRCC4 | during initial migration | 1999a and 1999b |
| | | | phase | |

By analogy with the immune system, it is proposed that the most efficacious mechanism to maintain lineage memory is DNA switching. To test this theory, the patterns of mobile-element-related repetitive sequences in the non-coding regions between the exons in multigene families of mobile element-related polypeptides were analyzed by searching data from both vertebrates and C. elegans. The search revealed numerous candidates for possible target sites of enzymes.

The role of DNA switch mechanisms in normal development arose at least two billion years ago in cyanobacteria (Haselkorn, 1992; Carrasco et al. 1994; Carrasco and Golden, 1995; Wolk, 1996; Canfield 1999). In some cells, these organisms excise circular DNA from germline DNA and generate somatic cells that can fix nitrogen for the use of the bacterial colony. The evidence is massive and impressive indicating that such genetic switches have been maintained as integral developmental control mechanisms in numerous living organisms during billions of years of evolution. In humans, however, evidence has been scant. The best-known example is in the immune system wherein circular DNA is excised as variable and constant regions or as heavy chain genes are rearranged.

The characterization of surface components of cells, on a tissue by tissue basis, would be a daunting task. The present invention, however, provides a rapid and unifying mechanism to characterize tissues and even individual cells, according to the genetic organization and the display, or the lack of display, of expression products of mobile element-related genes, alone or in combination with other cell surface molecules, including olfactory receptors and protocadherins.

This is the first suggestion that mobile element-related switching machinery may permanently switch DNA during development, resulting in altered DNA sequences in specific cell lineages. Such altered DNA sequences can be used to identify and characterize the specific cell lineages or cell type. The expression products resulting from such altered DNA may also be used to characterize or identify specific cell types. In a first embodiment, the invention provides a method for characterizing a developmental or lineage-specific cell type by analyzing nucleic acid switch patterns, other than immunoglobulin and/or T cell receptor nucleic acid switch patterns, or profiles and/or resulting gene products, other than immunoglobulins and/or T cell receptors. The method includes comparing the nucleic acid of the cell with nucleic acid from a corresponding germline cell or other cell, wherein a difference in the nucleic acid is indicative of a nucleic acid switch; and characterizing the cell in terms of developmental or lineage specific cell type.

A nucleic acid switch, as described herein, refers to a region of nucleic acid that is a “hot spot” for coordinating the removal of regions of nucleic acid. For example, an early DNA species may contain 5 kb of nucleic acid containing several sites for switching. A species of DNA that may be found later in a cell lineage may contain a ring of DNA that is excised once two “switches” or “hot spots” recombine, thereby eliminating a ring of DNA. Another species of DNA may contain further excisions at these hot spots or switches. A final DNA species may go from 5 kb to 3 kb after “switching” (e.g., a cell differentiation event). Profiles of cell types can be prepared based on the various patterns of nucleic acid switching that occur throughout development or lineage-specific decisions. Nucleic acid switching patterns are also found in various disease states, thereby providing diagnostic and prognostic profiles. Nucleic acid switching patterns are useful to broadly classify cell types and to specifically classify cell types, e.g., many types of stem cells or progenitor cells.

Nucleic acid to be detected in the methods of the invention may be present in extrachromosomal nucleic acid (e.g., in a “ring” structure that has been excised or in double minute chromosomes (DMs)); in cell-free nucleic acid samples; or in cell-associated nucleic acid samples, for example. Nucleic acid includes DNA or RNA or combinations thereof. Cells that may be identified by methods of the invention include any cell type, for example, stem cells, neuronal, epidermal, endodermal, mesodermal, hematopoietic, or non-germ cell stem cells, cells of the immune system, including B cell lineage cells, T cell lineage cells and other immune cells, provided the lineage and/or developmental stage is not determined based on immunoglobulin and/or T cell receptor nucleic acid switching or protein expression.

Genetic probes, such as DNA or RNA polynucleotides, can be used to identify the extent of genetic rearrangement in DNA associated with a switch region or a mobile element-related polypeptide or polynucleotides encoding such polypeptides, and thereby characterize or identify a population of cells. Detection of nucleic acid switches can be performed by standard methods such as size fractionating the nucleic acid. Methods of size fractionating the DNA and RNA are well known to those of skill in the art, such as by gel electrophoresis, including polyacrylamide gel electrophoresis (PAGE). For example, the gel may be a denaturing 7 M or 8 M urea-polyacrylamide-formamide gel. Size fractionating the nucleic acid may also be accomplished by chromatographic methods known to those of skill in the art. Both the native molecule and extrachromosomal molecules are detectable by methods know to those of skill in the art.

The detection of polynucleotides optionally can be performed by using radioactively labeled probes. Any radioactive label which provides an adequate signal can be employed. One of skill in the art can use Magnetic Resonance Imaging (MRI) to detect switches of the invention. Labels include binding agents, which can serve as a specific binding pair member for a labeled ligand, and the like. Labels include enzymes, radioisotopes, fluorescent compounds, colloidal metals, chemiluminescent compounds, phosphorescent compounds, and bioluminescent compounds, for example.

The labeled preparations are used to probe nucleic acid, for example, using Southern blot or northern blot hybridization techniques. Nucleic acid molecules obtained from samples are transferred to filters that bind polynucleotides. After exposure to the labeled nucleic acid probe, which will hybridize to nucleotide fragments containing target nucleic acid sequences, the binding of the radioactive probe to target nucleic acid fragments is identified by autoradiography (see Genetic Engineering, 1, ed. Robert Williamson, Academic Press (1981), pp. 72-81). The particular hybridization technique is not essential to the invention. Hybridization techniques are well known or easily ascertained by one of ordinary skill in the art. As improvements are made in hybridization techniques, they can readily be applied in the method of the invention.

The polynucleotides including switch regions or encoding polypeptides may be amplified before detecting. The term “amplified” refers to the process of making multiple copies of the nucleic acid from a single polynucleotide molecule. The amplification of polynucleotides can be carried out in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, other available DNA polymerases, polymerase muteins, reverse transcriptase, ligase, and other enzymes, including heat-stable enzymes (i.e., those enzymes that perform primer extension after being subjected to temperatures sufficiently elevated to cause denaturation). Suitable enzymes will facilitate combination of the nucleotides in the proper manner to form the primer extension products that are complementary to each mutant nucleotide strand. Generally, the synthesis will be initiated at the 3′ end of each primer and proceed in the 5′ to 3′ direction along the template strand, until synthesis terminates, producing molecules of different lengths. There may be amplification agents, however, that initiate synthesis at the 5′ end and proceed in the other direction, using the same process as described above. In any event, the method of the invention is not to be limited to the embodiments of amplification described herein.

One method of in vitro amplification that can be used according to this invention is the polymerase chain reaction (PCR) described in U.S. Pat. Nos. 4,683,202 and 4,683,195. The term “polymerase chain reaction” or “PCR” refers to a method for amplifying a DNA base sequence using a heat-stable DNA polymerase and two oligonucleotide primers, one complementary to the (+)-strand at one end of the sequence to be amplified and the other complementary to the (−)-strand at the other end.

Primers used according to the method of the invention are complementary to each strand of nucleotide sequence to be amplified. The term “complementary” means that the primers must hybridize with their respective strands under conditions that allow the agent for polymerization to function. In other words, the primers that are complementary to the flanking sequences hybridize with the flanking sequences and permit amplification of the nucleotide sequence. Preferably, the 3′ terminus of the primer that is extended has perfectly base paired complementarity with the complementary flanking strand.

Those of ordinary skill in the art will know of various amplification methodologies that can also be utilized to increase the copy number of target nucleic acid. The polynucleotides detected in the method of the invention can be further evaluated, detected, cloned, sequenced, and the like, either in solution or after binding to a solid support, by any method usually applied to the detection of a specific nucleic acid sequence such as another polymerase chain reaction, oligomer restriction (Saiki et al., BioTechnology 3:1008-1012 (1985)), allele-specific oligonucleotide (ASO) probe analysis (Conner et al., Proc. Natl. Acad. Sci. USA 80: 278 (1983), oligonucleotide ligation assays (OLAs) (Landegren et al., Science 241: 1077 (1988)), RNAse Protection Assay and the like. Molecular techniques for DNA analysis have been reviewed (Landegren et al, Science, 242: 229-237 (1988)). Following DNA amplification, the reaction product may be detected by Southern blot analysis, without using radioactive probes. In such a process, for example, a small sample of DNA containing a the polynucleotides obtained from the cells or tissue or subject are amplified, and analyzed via a Southern blotting technique. The use of non-radioactive probes or labels is facilitated by the high level of the amplified signal. In a one embodiment of the invention, one nucleoside triphosphate is radioactively labeled, thereby allowing direct visualization of the amplification product by autoradiography. In another embodiment, amplification primers are fluorescent labeled and run through an electrophoresis system. Visualization of amplified products is by laser detection followed by computer assisted graphic display. Simple visualization of a gel containing the separated products may be utilized to determine the presence of a polynucleotide. However, other methods known to those skilled in the art may also be used, for example scanning densitometry, computer aided scanning and quantitation.

Polynucleotides encoding mobile element-related polypeptides can be identified by nucleic acid hybridization techniques. In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter. An example of progressively higher stringency conditions is as follows: 2× standard saline citrate (SSC)/0.1% sodium dodecyl sulfate (SDS) at about room temperature (hybridization conditions); 0.2× SSC/0.1% SDS at about room temperature (low stringency conditions); 0.2× SSC/0.1% SDS at about 42° C. (moderate stringency conditions); and 0.1× SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Biological chips or arrays are useful in a variety of screening techniques for obtaining information about nucleic acid switching profiles or patterns or mobile element-related polypeptide display on cell surfaces. Arrays of nucleic acid probes can be used to extract sequence information from, for example, nucleic acid samples. The samples are exposed to the probes under conditions that allow hybridization. The arrays are then scanned to determine to which probes the sample molecules have hybridized. One can obtain sequence information by careful probe selection and using algorithms to compare patterns of hybridization and non-hybridization. This method is useful for sequencing nucleic acids, as well as sequence checking, and further is useful in diagnostic screening for genetic diseases or for the presence and/or identity of a particular pathogen or a strain of pathogen. For example, there are various strains of HIV, the virus that causes AIDS, some of which have become resistant to current AIDS therapies. Diagnosticians can use DNA arrays to examine a nucleic acid sample from the virus to determine what strain it belongs to. In the same way, the genetic fingerprint including nucleic acid switches or polynucleotides encoding mobile element-related polypeptides, can be compared with nucleic acid samples extracted from different cell samples, e.g., to identify cell lineages.

The biological chip plates used in the methods of this invention include biological chips. The array of probe sequences can be fabricated on the biological chip according to the pioneering techniques disclosed in U.S. Pat. No. 5,143,854, PCT WO 92/10092, PCT WO 90/15070, or U.S. Pat. Nos. 5,856,101; 6,420,169; and 6,284,460. The combination of photolithographic and fabrication techniques may, for example, enable each probe sequence (“feature”) to occupy a very small area (“site” or “location”) on the support. In some embodiments, this feature site may be as small as a few microns or even a single molecule. For example, a probe array of 0.25 mm2 (about the size that would fit in a well of a typical 96-well microtiter plate) could have at least 10, 100, 1000, 104, 105 or 106 features. In an alternative embodiment, such synthesis is performed according to the mechanical techniques disclosed in U.S. Pat. No. 5,384,261, incorporated herein by reference. Sensitive analysis of mobile element-related nucleic acid can also be performed as described by Clinical Microsystems, using AC to detect minute changes in electron flow in dsDNA after DNA fragments hybridize to an array of DNA on a chip.

In further embodiments, an oligonucleotide derived from any of the polynucleotide sequences described herein may be used as a target in a microarray. The microarray can be used to monitor the expression level of large numbers of genes simultaneously (to produce a transcript image), and to identify genetic variants, mutations and polymorphisms. This information will be useful in determining gene function, understanding the genetic basis of disease, diagnosing disease, and in developing and monitoring the activity of therapeutic agents (Heller, R. et al. (1997) Proc. Natl. Acad. Sci. 94:2150-55).

The microarray is preferably composed of a large number of unique, single stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20-25 nucleotides in length. For a certain type of microarray, it may be preferable to use oligonucleotides which are only 7-10 nucleotides in length. The microarray may contain oligonucleotides which cover the known 5′ sequence, or 3′, sequence, sequential oligonucleotides which cover the full length sequence; or unique oligonucleotides selected from particular areas along the length of the sequence. Polynucleotides used in the microarray may be oligonucleotides that are specific to a gene or genes of interest in which at least a fragment of the sequence is known or that are specific to one or more unidentified cDNAs which are common to a particular cell type, developmental or disease state.

Cells which contain the nucleic acid sequence including DNA switches or encoding one or more mobile element-related polypeptide may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein.

The presence of polynucleotide sequences including switch regions or encoding mobile element-related polypeptides can be detected by DNA-DNA or DNA-RNA hybridization or amplification using probes or fragments or fragments of polynucleotides. Nucleic acid amplification based assays involve the use of oligonucleotides or oligomers based on the sequences encoding mobile element-related polypeptides to detect cells containing DNA or RNA.

A biological sample can be obtained from any bodily fluids (such as blood, urine, saliva, phlegm, gastric juices, etc.), cultured cells, biopsies, or other tissue preparations. A detection system may be used to measure the absence, presence, and amount of hybridization or binding for all of the distinct molecules simultaneously. This data can be used for large scale correlation studies on the sequences, mutations, variants, or polymorphisms among samples.

A variety of protocols for detecting and measuring the expression of mobile element-related polypeptides, using either polyclonal or monoclonal antibodies specific for the protein are known in the art. Examples include enzyme-linked immunosorbent assay (ELISA), radioimmunoassay (RIA), and fluorescence activated cell sorting FACS). A two-site, monoclonal-based immunoassay utilizing monoclonal antibodies reactive to two non-interfering epitopes on mobile element-related polypeptides can be used, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding mobile element-related polypeptides include oligolabeling, nick translation, end-labeling or PCR amplification using a labeled nucleotide. Alternatively, the sequences encoding mobile element-related polypeptides, or any fragments thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia and Upjohn, (Kalamazoo, Mich.); Promega (Madison Wis.); and U.S. Biochemical Corp., Cleveland, Ohio). Suitable reporter molecules or labels, which may be used for ease of detection, include radionuclides, enzymes, fluorescent, chemiluminescent, or chromogenic agents as well as substrates, cofactors, inhibitors, magnetic particles, and the like.

Binding agents such as ligands or antibodies, specific for such mobile element-related polypeptides, are used for such identification and characterization. The preparation of polyclonal antibodies is well-known to those skilled in the art. See, for example, Green et al., “Production of Polyclonal Antisera” in Immunochemical Protocols (Manson, ed.), pages 1-5 (Humana Press 1992); Coligan et al., “Production of Polyclonal Antisera in Rabbits, Rats, Mice and Hamsters” in Current Protocols In Immunology, section 2.4.1 (1992), which are hereby incorporated by reference.

One embodiment of the invention provides a method of obtaining a specific cell type or lineage. The method includes obtaining a sample of cells, contacting the cells with an agent, such as a nucleic acid probe for identifying nucleic acid switches or an antibody or a ligand specific for a mobile element-related polypeptide or polynucleotide indicative of a particular cell type such that the antibody or ligand binds to a cell in the sample, and separating the cell that is bound by the antibody or ligand from the sample, thereby obtaining a population of a specific cell type or lineage. The cell population may be further purified by selecting for cells by expression of at least one additional marker associated with a specific cell type. For example, the additional marker may include CD-34, Thy-1, rho, Cdw109, protocadherins, and cell adhesion molecules, such as O-CAM, alone or in combination with other cell surface receptors. The method of the invention includes identifying a cell type by detecting expression of at least one mobile element-related polypeptide, wherein the presence or absence of the mobile element-related polypeptide is indicative of a cell type or lineage. In addition to analyzing the presence of such mobile element-related polypeptides on the cell surface, one can also analyze the genetic fingerprint of the cell, e.g., identify changes in DNA as a result of switching or detect the presence or absence of RNA transcripts. The preparation of monoclonal antibodies likewise is conventional. See, for example, Kohler and Milstein, Nature 256:495 (1975); Coligan et al., sections 2.5.1-2.6.7; and Harlow et al., Antibodies: A Laboratory Manual, page 726 (Cold Spring Harbor Pub. 1988), which are hereby incorporated by reference.

The term “antibody” as used in this invention includes intact molecules as well as fragments thereof, such as Fab, F(ab′)2, and Fv which are capable of binding to an epitopic determinant present in Bin1 polypeptide. Such antibody fragments retain some ability to selectively bind with its antigen or epitope. As used in this invention, the term “epitope” refers to an antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibodies which bind to mobile element-related polypeptides can be prepared using an intact polypeptide or fragments containing small peptides of interest as the immunizing antigen. For example, it can be desirable to produce antibodies that specifically bind to the extracellular loop, or the N-terminal or C-terminal or other domains of a mobile element-related polypeptide. The polypeptide or peptide used to immunize an animal which is derived from translated cDNA or chemically synthesized which can be conjugated to a carrier protein, if desired. Such commonly used carriers which are chemically coupled to the immunizing peptide include keyhole limpet hemocyanin (KLH), thyroglobulin, bovine serum albumin (BSA), and tetanus toxoid.

In another embodiment, nucleic acid patterns or profiles or patterns of antibody binding by antibodies which specifically bind mobile element-related polypeptides can be used for the diagnosis of conditions or diseases characterized by expression of specific switches or mobile element-related polypeptides, or in assays to monitor patients being treated. Diagnostic assays for mobile element-related polypeptides include methods which utilize nucleic acid probes or an antibody and a label to detect switch patterns or mobile element-related polypeptide patterns in human body fluids or extracts of cells or tissues. The antibodies may be used with or without modification, and may be labeled by joining them, either covalently or non-covalently, with a reporter molecule. A wide variety of reporter molecules which are known in the art may be used, several of which are described above.

A variety of protocols including ELISA, RIA, and FACS for measuring antibody-protein interactions are known in the art and provide a basis for diagnosing levels of polypeptide expression. Normal or standard values for mobile element-related polypeptides expression are established by combining body fluids or cell extracts taken from normal mammalian subjects, preferably human, with antibody under conditions suitable for complex formation. The amount of standard complex formation may be quantified by various methods, but preferably by photometric, means. Quantities of mobile element-related polypeptides expressed in subject, control and disease, samples from biopsied tissues are compared with the standard values. Deviation between standard and subject values establishes the parameters for diagnosing disease.

In another embodiment of the invention, the polynucleotides encoding mobile element-related polypeptides may be used for diagnostic purposes. The polynucleotides that can be used include oligonucleotide sequences, complementary RNA and DNA molecules. The polynucleotides can be used to detect and quantitate gene expression in biopsied tissues in which expression of mobile element-related polypeptides may be correlated with disease. The diagnostic assay can be used to distinguish between absence, presence, and excess expression of mobile element-related polypeptides, and to monitor regulation of mobile element-related polypeptides levels during therapeutic intervention.

In one aspect, hybridization with PCR probes which are capable of detecting polynucleotide sequences, including genomic sequences, encoding mobile element-related polypeptides or closely related molecules, or switches may be used to identify nucleic acid sequences which encode mobile element-related polypeptides. The specificity of the probe, whether it is made from a highly specific region, e.g., 10 unique nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding mobile element-related polypeptides, alleles, or related sequences.

In another embodiment of the invention, the nucleic acid sequences which encode mobile element-related polypeptides may also be used to generate hybridization probes which are useful for mapping the naturally occurring genomic sequence and for detecting differences in the sequence that might be indicative of a lineage. The sequences may be mapped to a particular chromosome, to a specific region of a chromosome or to artificial chromosome constructions, such as human artificial chromosomes (HACs), yeast artificial chromosomes (YACs), bacterial artificial chromosomes (BACs), bacterial P1 constructions or single chromosome cDNA libraries as reviewed in Price, C. M. (1993) Blood Rev. 7:127-134, and Trask, B. J. (1991) Trends Genet. 7:149-154.

Fluorescent in situ hybridization (FISH as described in Verma et al. (1988) Human Chromosomes: A Manual of Basic Techniques, Pergamon Press, New York, N.Y.) can be correlated with other physical chromosome mapping techniques and genetic map data. Examples of genetic map data can be found in various scientific journals or at Online Mendelian Inheritance in Man (OMIM). Correlation between the location of the gene encoding mobile element-related polypeptides on a physical chromosomal map and a specific disease, or predisposition to a specific disease, may help delimit the region of DNA associated with a particular cell lineage. The nucleotide sequences of the subject invention may be used to detect differences in gene sequences between cell lineages for diagnostic, therapeutic or other applications as discussed throughout the specification.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

The following is an example of how this might be used for cancer therapy. There are a number of molecules that, in isolation, are non-toxic. When combined with other non-toxic molecules, the combination is toxic. Imagine one such molecule, targeted by means of, say, an antibody to a specific mobile element-related polypeptide characteristic of a specific lineage (e.g., the particular B-cell lineage associated with a patient's lymphoma). The molecule would be drawn not only to the specific mobile element-related polypeptide on those B-lymphoma cells (which is what you want) but also to other sites within the body (which you don't want). Then, if a second molecule (non-toxic unless combined with the first), likewise targeted to another surface determinant of the lymphoma, is introduced, it finds the lymphoma cells and other, different cells. Only the cells that are targets for both molecules (lymphoma cells) are delivered a toxic dose, thereby reducing non-specific toxicity of the cancer drugs.

Such a scheme also can be used in genetic therapy approaches, with specific genetic sequences carrying enabling and coding functions delivered independently to different molecules of the mobile element-related polypeptide address, so that the genetic therapy is targeted appropriately. Also, complementary strands of RNA could be delivered independently in order to inhibit specific genes, since it is known that dsRNA can block gene transcription in ways that ssRNA (in antisense orientation) does not block transcription.

In another embodiment, competitive screening assays can be used in which ligands or other molecules capable of binding mobile element-related polypeptides specifically compete with a test compound or ligand for binding mobile element-related polypeptides. In this manner, the ligand or test compound can be used to detect the presence of any molecule which shares one or more antigenic or binding determinants (i.e., epitopes) with mobile element-related polypeptides. In additional embodiments, the nucleotide sequences that encode mobile element-related polypeptides can be used in any molecular biology techniques that have yet to be developed, provided the new techniques rely on properties of nucleotide sequences that are currently known, including, but not limited to, such properties as the triplet genetic code and specific base pair interactions.

Progenitor cells that are committed to being a specific cell type, but still capable of further differentiation, including totipotential and pluripotential progenitor cells such as germ cells and mesenchymal stem cells, respectively, and more tissue specific progenitor cells such as chondrocytes, display specific mobile element-related polypeptides that are characteristic of each lineage. The cell surface display of these codes can be used to identify cell-specific lineages. The importance of progenitor cells has been recognized already in some fields of therapy, including tissue engineering, bone marrow ablation therapies, etc. For example, progenitor cell lines isolated from bone marrow or circulating blood have been used to re-populate the hematopoietic system in individuals whose bone marrow is ablated and then reconstituted in bone marrow transplantation procedures. Certain neurological defects, such as Parkinson's disease and others, have been cured or ameliorated through the transplantation of fetal or immature tissues. These results have been made possible by a re-growth and differentiation of tissue originating from progenitor cells.

Utilizing mobile element-related polypeptides that characterize the surface of specific progenitor cells, these cells can be isolated by a number of cell selection techniques (FACS, immunomagnetic beads, others). Such selection techniques can include both positive selection, for example identifying and removing the cell of interest from a population, as well as negative selection, removal of the positive cells from the population leaving only the negative cells. Negative selection may prove useful in isolating cells that have yet to differentiate sufficiently to express a particular mobile element-related polypeptide. Further, an understanding of both the surface characteristics and also the genetic switching processes relating to mobile element-related polypeptides will be useful in the development of cell culture techniques to maintain and propagate such cells in their progenitor state. Purified progenitor cells are likely to become important therapeutic moieties in the treatment of disease and deficiencies.

Data obtained by searching the genomic databases have provided evidence suggesting that the mobile element-related polypeptides may indeed be used in a combinatorial array with other cell surface address molecules during the assembly of many tissues. Such molecules therefore have many of the properties expected for area code molecules.

To determine variations in mobile element-related polypeptides or in polynucleotides encoding them, homology or identity is often measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that have a specified percentage of amino acid residues or nucleotides, which can be 100%, respectively, that are the same when compared and aligned for maximum correspondence over a comparison window or designated region as measured using any number of sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from about 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith and Waterman, Adv. Appl. Math. 2:482, 1981, by the homology alignment algorithm of Needleman and Wunsch, J. Mol. Biol 48:443, 1970, by the search for similarity method of person and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals and Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction and Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF.

Such alignment programs can also be used to screen genome databases to identify polynucleotide sequences having substantially identical sequences. A number of genome databases are available, for example, a substantial portion of the human genome is available as part of the Human Genome Sequencing Project (J. Roach, using hypertext transfer protocol “http”, at the URL “weber.u.Washington.edu/˜roach/human_genome_progress2.html”; Gibbs, 1995). At least twenty-one other genomes have already been sequenced, including, for example, M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997), and D. melanogaster (Adams et al., 2000). Significant progress has also been made in sequencing the genomes of model organism, such as mouse, C. elegans, and Arabadopsis sp. Several databases containing genomic information annotated with some functional information are maintained by different organization, and are accessible via the internet, for example, using “http”, at the URL “wwwtigr.org/tdb”; on the world wide web, at URL “genetics.wisc.edu”; at URL “genome-www.stanford.edu/˜ball”; at URL “hiv-web.lanl.gov”; on the world wide web, at URL “ncbi.nlm.nih.gov”; on the world wide web, at URL “ebi.ac.uk:; at URL “Pasteur.fr/other/biology”; or on the world wide web at URL “genome.wi.mit.edu”.

One example of a useful algorithm is BLAST and BLAST 2.0 algorithms, which are described in Altschul et al., Nucl. Acids Res. 25:3389-3402, 1977, and Altschul et al., J. Mol. Biol. 215:403-410, 1990, respectively. Software for performing BLAST analyses is publicly available through the National Center for Biotechnology Information (on the world wide web, at URL “ncbi.nlm.nih.gov”). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=-4 and a comparison of both strands. For amino acid sequences, the BLASTP program uses as defaults a word length of 3, and expectations (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=-4, and a comparison of both strands.

The BLAST algorithm also performs a statistical analysis of the similarity between two sequences (see, e.g., Karlin and Altschul, Proc. Natl. Acad. Sci. USA 90:5873, 1993). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001.

The following examples are intended to illustrate but not limit the invention.

### EXAMPLE 1

Internet Grateful Med and SciSearch (ISI) databases were used for retrieval of bibliographic information. Large numbers of references including abstracts were downloaded into Procite 5 (ISI) for further searching and analysis locally as well as for formatting references. The online resources available through The National Center for Biotechnology Information (world wide web, at URL “ncbi.nlm.nih.gov”) were used extensively in this work. The information that is reported in Table 1 was obtained by searching the dbEST database using the text strings shown in Table 1. The quality of the sequence data varied widely as is normal for the expressed sequence tags. Nevertheless, it was clear that this approach provided a great deal of useful information on the expression of mobile element-related polypeptide genes in a large number of different tissues. Only the retrieved sequences that are related to known mobile element-related polypeptides are included in Table 1. Other informative searches used known amino acid sequences of specific mobile element-related polypeptides from various species to retrieve expressed sequence tags. For these studies, BLAST 2.0 (Gapped BLAST and Graphical Viewer) with the advanced BLAST option was used. The TBLASTN program was used to search the dbEST database.

Typically, nucleic acid sequence information for a desired mobile element-related polypeptide or other protein can be located in one of several public databases, e.g., GenBank, EMBL, SwissProt, and PIR, or in biological related journal publications. Thus, one of skill in the art would have access to nucleic acid sequence information for virtually all known genes. Those of skill in the art can either obtain the corresponding nucleic acid molecule directly from a public depository or the institution that published the sequence. Alternatively, once the nucleic acid sequence encoding a desired protein has been ascertained, the skilled artisan can employ routine methods, e.g., polymerase chain reaction (PCR) amplification to isolate the desired nucleic acid molecule from the appropriate nucleic acid library. Thus all known nucleic acids encoding proteins of interest, e.g., mobile element-related polypeptides, are available for use in the methods and products described herein.

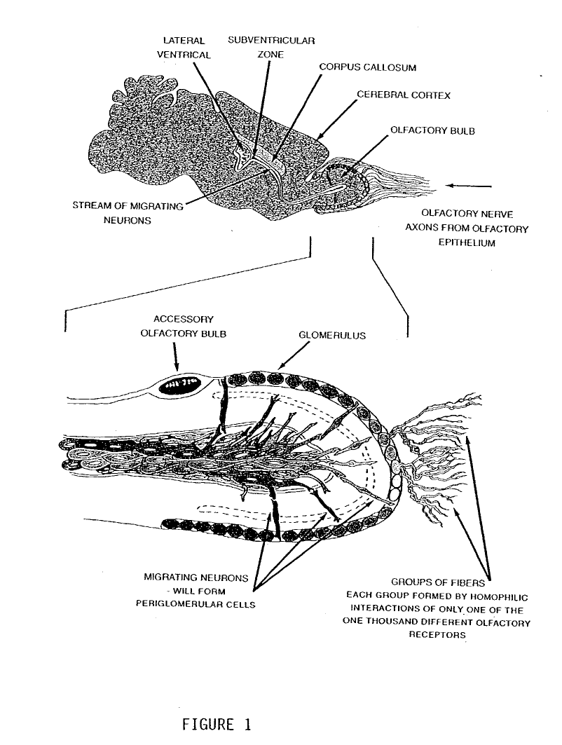

It was the analysis of the enormous precision for assembly of the olfactory system that revealed the identity of the key proposed area code molecules and gave clues as to their mode of action. Recent research has shown that the olfactory receptors function not only as odor detectants, but also play an important role in axonal targeting as their processes extend from the olfactory epithelium to specific glomeruli in the olfactory bulb of the brain (Ressler et al. 1994; Singer et al. 1995; Mombaerts, 1996; MOMBAERTS et al. 1996; see FIG. 1). There are one thousand or so different genes that code for olfactory receptors. About the same number of glomeruli are arranged in precise, topologically ordered arrays on both sides of the olfactory bulbs. These glomeruli serve as highly specific targets for the growth cones of the olfactory neurons, each expressing a single receptor gene. The fact that olfactory receptors not only interact with odorants in the nose but also are also capable of assisting in highly specific axonal targeting reveals a dual function of great interest. Thus they bear the hallmarks of the proposed cell-surface address molecules.

There are many molecules in addition to the olfactory and VNO receptors that play an important part in cell surface recognition. One example of an area code molecule is O-CAM, a member of the immunoglobulin supergene family (Yoshihara, 1997; Yoshihara et al. 1997). O-CAM is expressed on a subset of olfactory nerve axons that extend from the four zones of the olfactory epithelium to the specific zones of glomeruli in the olfactory bulb. This molecule is expressed on axons originating in three of the four zones of the olfactory epithelium and on one of the two zones from the VNO region. O-CAM thus seems to provide an excellent candidate for an address molecule coding for geographic regions rather than for a specific cellular addresses. It is predicted that other, probably related receptors will be found on zones in which O-CAM is absent and that these will form part of a combinatorial code.

Another exceptionally interesting example of address molecules is the large family of protocadherins that are differentially expressed on neurons and other cells and that aid in highly specific cell-cell recognition. Protocadherins are expected to play a role as area code molecules second in specificity only to the seven-transmembrane/olfactory receptors.

The role of area code molecules in the assembly of the olfactory bulb as a model for the assembly of the entire embryo: Olfactory receptors help incoming axons home to their targets in the olfactory bulb with remarkable accuracy, but how are the topologically precise targets of these olfactory axons, the olfactory bulb itself, assembled? Several research groups agree that olfactory neurons expressing the same olfactory receptor, from among the one thousand or so total receptors, converge on a single pair of glomeruli in each of the two target areas on an olfactory bulb. A logical consequence of this fact is that each glomerulus in one of the bilaterally symmetrical target structures has a unique address on the fixed topological map. There are about one thousand distinct addresses in each map. Furthermore, the maps are the same in each of the inbred individuals and they are believed to be “hardwired” by genetic programs that control brain development. It has been determined that the targets are established during embryogenesis. When the growth cones of olfactory neurons start entering the olfactory bulb, the targets await. It follows that the assembly of this target structure must itself use a very sophisticated molecular addressing system during embryogenesis and then display molecules that provide the topologically precise, distinct targets for olfactory nerve growth cones.

The subventricular zone, a considerable distance posterior to the region where the olfactory bulb is formed, is the birthplace of neuronal precursor cells that are destined to form the olfactory bulb. As such cells are born they begin migrating along a narrow tube-like pathway. The migrating spindle-shaped cells remain in contact with neighboring cells in front, beside and behind and migrate as a stream only a few cells in diameter. Cell division continues while they migrate and maintain contacts. As cells in this stream reach the inner region of the developing olfactory bulb some form granule cells but many change directions and move outward toward their final positions near the surface of the bulb and become periglomerular cells. The dendrites of these cells become targets for the growth cones of olfactory cell axons that form synapses with them. A required consequence of this behavior seems to be that this pattern of cell generation and migration relates directly to the setting up of specificity of the target receptor(s) that each glomerulus will ultimately express. This process forms the remarkably precise and bilaterally symmetrical topological map of future targets for the growth cones extending from olfactory neurons born in the olfactory epithelium to the glomeruli in the olfactory bulb.

Olfactory receptors play a key and proven role as address molecules targeting the glomeruli. But what molecules form the targets and what known gene families might code for such receptors? Is it reasonable to suppose that a totally different mechanism is used as cells there migrate to form that extraordinarily precise target structure, the olfactory bulb? Why not use the same families of genes, again in a combinatorial code, for the formation of this neural structure? What molecular codes are used to assemble other parts of the brain by nearby cells in the fate map of the subventricular zone? What about other parts of the brain and, indeed, other regions of the embryo? It seems logical to propose that olfactory and VNO receptors, as well as protocadherins are expressed throughout the brain and embryo and serve as area code molecules during embryonic development. As disclosed herein, a search of the expressed sequence database (dbEST) revealed that olfactory receptors and related molecules are expressed in essentially all tissues examined. Additional recent results support the notion that these receptors are indeed expressed outside of the olfactory system. A separate search of dbEST revealed that members of the large protocadherin multigene family are also expressed in all tissues examined. Thus, it is reasonable to consider that the principle of gradients of receptor affinities can be part of a general mechanism for cell sorting and assembly of embryos.

Gradients of receptor affinities: a molecular model for assembly of complex organs by means of area code molecules. As discussed above, the possibility that members of the olfactory and VNO receptor families, as well as protocadherins, are expressed in the cells that form the target arrays in the olfactory bulb, is considered. In this scenario, a homophilic molecular interaction of these receptors with themselves provides the required specificity for both migration and recognition of their specific target. How then could cells interact with their neighbors in such a way as to form the precise topological map of cells expressing target receptors? One intriguing possibility is suggested by the structure of the olfactory receptors themselves and by certain interesting patterns in which these structures are arrayed in the target maps. All of the olfactory receptors contain seven helical domains that traverse the membrane and arrange themselves so as to form a pocket at the cell surface. Studies have shown that these pockets provide specific sites for binding ligands. Consider the notion that the binding sites provide the required specificity for both homophilic and heterophilic interactions of each of these classes of receptors. Homophilic interactions could account for the target specificity known to occur as the olfactory axons seek specific glomeruli in the olfactory bulb and for the specificity of the fasciculation of axons expressing the same receptor. But how is the specificity of cell migration and bulb assembly explained? A possible hint derives from the observation that olfactory receptors with an unusual type of extracellular loop structure cluster together in both the olfactory epithelium and in the target bulb structure. Indeed, numerous studies suggest that glomeruli are arranged with receptors of similar structure displayed on adjacent glomeruli and within a specific region of the olfactory bulb. It seems possible that receptors differing only slightly in the amino acid sequence of the binding sites responsible for homophilic interactions could still interact with relatively high affinity. The binding constant difference could serve to guide neighbors to each other. Other adjacent cells could again have receptors with close but lower affinity. In this manner a type of affinity gradient could be established that could help explain the relationships maintained among cells as they migrate and assemble the target map in the olfactory bulb. Such a gradient of receptor affinities would also aid the growth cones of olfactory neurons as they both fasciculate with themselves and seek their targets in the bulb.

The protocadherins are excellent candidates for a somewhat less specific role in this process. They might, for example, provide a similar but broader specificity. They too have been shown to interact homophilically. Furthermore, the large number of very similar sequences of binding regions in this multigene family suggests that they too might display heterophilic interactions. While these suggestions of a gradient in receptor affinities that is recognized by cells to aid them in seeking their targets are clearly hypothetical at this time, mechanisms with at least this degree of address-coding specificity are required if the precision with which migrating cells and their processes assemble organisms is to be explained.

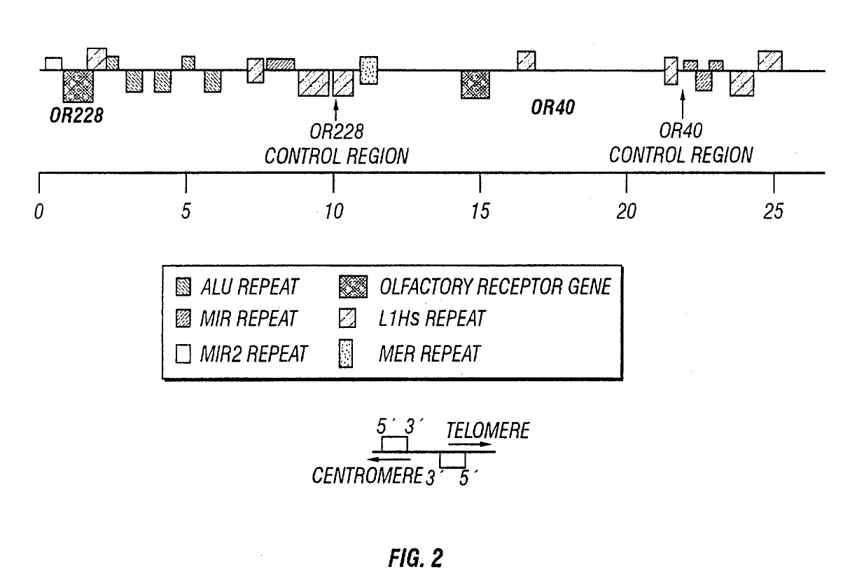

What sort of orderly genetic programs are sophisticated enough to generate and maintain one thousand or more cells, each expressing one receptor gene?: Elaborate genetic controls must function to maintain the expression of a single, specific olfactory receptor gene in each of the olfactory stem cells and in its daughter olfactory neurons as they continue to be born throughout life. Furthermore, these controls must allow the expression of only one of the two alleles present in each cell. The complexity of this genetic problem is very reminiscent of the situation seen in the immune system where sophisticated alterations are made in the germline DNA as specific B or T cells are generated. There, too, only a single allele is expressed in each cell. The altered DNA sequences are replicated for the life of a stem cell thus accounting for the lineage memory. Genetic switching therefore remains an attractive aspect of the area code hypothesis, particularly for the control of the expression of the protocadherin and olfactory receptors discussed here. Indeed, it is extremely difficult to imagine that a mechanism utilizing only transcription factors et cetera is capable of mimicking the immune system's single-allele expression and stem cell specific receptor expression. The recent discovery that the protocadherin proteins appear to be controlled and formed by splicing one of a large number of variable regions in the genome to a common region (Obata et al. 1995; Kai et al. 1997; Kohmura et al. 1998; Mombaerts, 1999; Serafini, 1999; Wu and Maniatis, 1999; Chun, 1999; see FIG. 2a) adds support for the view that recombinases and reverse transcriptases switch genes in families other than those of the immune system. Another recent publication demonstrated that, in zebra fish, the rag 1 recombinase is expressed in the olfactory epithelium as well as in tissues in which common and variable genes are switched in the immune system, thus adding further support to the notion of wider use of these mobile-element-related mechanisms in development.

There are a number of other studies that show remarkable tissue specificity in the expression of such elements. In both mice and humans numerous retro-elements are individually expressed in a tissue-specific way, each under the control of a factor appropriate for the tissue in which it is expressed. For example, EGF can stimulate the expression of a retroelement with the appropriate target sequence in its LTR. Corticosteroids stimulate the expression of different retroelements in the adrenal glands. The LTR control sequences differ appropriately in a number of different tissues where other growth factors and hormones stimulate the expression of specific retroelements. What evolutionary pressures could explain these results? It is assumed that these mobile elements provide a useful function when they are expressed in such a controlled and tissue-specific way.

Developmentally timed expression of env and other endogenous retroviral products have been noted with great interest. The discovery of the expression of env gene products on mouse and human unfertilized oocytes, and the diminution of this expression after fertilization, raises the intriguing possibility that these gene products are involved in sperm-egg binding and fertilization.

Another remarkable study examined the expression of more than fifteen mobile element-related genes in Drosophila tissues. In situ hybridization revealed RNA expression patterns that differed dramatically for almost all elements. The patterns are complex and definitive, reminiscent of the patterns of homeobox gene expression. The patterns of mobile-element-related RNA expression evolve in time and space in a reproducible manner as embryonic development proceeds. Again, how did this extreme control evolve if there is no function and hence no selective survival value for these genes?

There are numerous examples of critical functions that are performed in diverse organisms by mobile-element genes. The ciliates use recombinases etc. to radically process the DNA of the germline micronucleus as the somatic macronucleus is created. The nematode, Ascaris uses similar programmed expression of transposases, etc., to convert the germline chromosomes to radically different somatic chromosomes. Drosophila uses two non-LTR retrotransposons (HeT-A and TART) to maintain its telomeres. There are a number of reviews of this subject that provide many more examples of useful and programmed functions of mobile-element-related genes in organisms. Perhaps the genes found in our searches of the EST databases also perform important functions in DNA processing and cell addressing. On the other hand, there can be no doubt that uncontrolled transposition of some elements also occur. These are not mutually exclusive processes. Indeed the mobility, combined with important cellular and developmental functions, provides an important insight into mechanisms of evolution.

### EXAMPLE 2

### Olfactory Neurons Each Express a Single Receptor, and Use that Receptor to Target a Specific Pair of Bilaterally Symmetrical Glomeruli

Recent research including the elegant experiments by Mombaerts et al. (9,10) has shown that the olfactory receptors themselves do in fact play an important role in axonal targeting as their processes extend from the olfactory epithelium to specific glomeruli in the olfactory bulb. Neurons that express the same receptor gene but are dispersed in the olfactory epithelium target their processes to a single pair of bilaterally symmetrical glomeruli (11,12; see FIG. 1). There are one thousand or so different genes that code for olfactory receptors. About the same number of glomeruli are arranged in a precise, topologically ordered array in each of the two sides of the olfactory bulb. These serve as highly specific targets for the growth cones of the olfactory neurons, each expressing a single receptor gene. Because these olfactory receptors bear the hallmarks of the proposed area code molecules, it seemed appropriate to ask if they might be expressed in other parts of the developing embryo (and adult) as expected for such molecular codes.

A search of the genome and literature databases revealed a remarkable number of examples of these genes expressed in tissues other than the olfactory system. Axons expressing VNO receptors are believed to target the accessory olfactory bulb with similar high precision and they too are assumed to play a role in cell targeting.

### EXAMPLE 3

### Expression of Members of these Families of Receptors in Tissues other than the Olfactory Epithelium

Expressed sequence tags are being entered into the dbEST database at a rapid rate and now represent an important new resource for the study of gene expression. The cDNA samples used for these sequencing studies are obtained from a wide variety of tissues, developmental stages and organisms. The data vary in quality but nevertheless provide a rich source of information. A search of dbEST revealed many examples of the expression of olfactory receptor genes expressed in tissues other than the olfactory system. Surprisingly, the identified genes are expressed in liver, lung, colon, testis, ovary, uterus, prostate, thyroid, brain and many other tissues and tumors. In addition, a search of the bibliographic databases revealed several publications dealing with the expression of olfactory receptors in a few tissues (13-15).

The original area code paper reviews a number of systems in which cell migration plays a role in organogenesis. The embryonic heart is a particularly interesting example of an organ that is assembled using migrating cells that coalesce and construct the tissue with great precision. In pursuing the notion that serpentine receptors can act as receptors in an area code system, it was gratifying that the searches of dbEST revealed that specific olfactory receptors are indeed expressed in the embryonic heart. A publication was also found that provides further evidence for such expression (13). One olfactory receptor, OL1, was studied in detail and the data, including in situ hybridization studies, seem very convincing. The authors further stated that other olfactory receptors are also expressed in the embryonic heart but give no data. It will be most interesting to learn the extent, timing, and topography of the expression of these receptors in the embryonic heart and also in the many other organs where they are expressed.

The widespread expression of members of the serpentine receptor family in numerous organ systems obviously supports the hypothesis that the receptors perform functions other than the recognition of olfactants. Since these receptors play a dual role as receptors for molecules in the olfactory epithelium and as cell surface addressing molecules that aid in the assembly of the olfactory bulb, one obvious notion is that they may also play a dual role in other parts of the embryo. The possibility of the combined functions of cell-cell recognition and organ construction, and also as cell surface receptors for many classes of small molecules, represents an extremely provocative concept when considering the roles of these very large families of genes. Another surprising consequence of this notion is that some of the very widely expressed receptors of the calcium sensing and metabotrophic glutamate families (found in the. VNO/accessory olfactory system) may also have dual functions and thus play a role in cellular addressing during development. One would certainly not anticipate or postulate a dual role for these receptor classes if members of these families were not functional in the VNO olfactory system as receptors for pheromones and other small molecules and for targeting the accessory olfactory bulb (16-20).

Assembly of the Olfactory bulb: A Model for other Parts of the Brain and Embryo. As discussed above, several research groups agree that olfactory neurons expressing the same serpentine receptor, from among the one thousand or so total receptors, converge on a single pair of glomeruli in the olfactory bulb. A logical consequence of this fact is that each glomerulus in one of the bilaterally symmetrical olfactory lobes has a unique address on the fixed topological map of the olfactory bulb. There are about one thousand distinct addresses in each lobe. Furthermore, the maps are the same in each of the inbred individuals and they are believed to be “hardwired” by genetic programs that control development. It has been determined that the targets are established during embryogenesis. When the growth cones of olfactory neurons start entering the olfactory bulb, the targets await. It follows that the assembly of this target structure must itself use a very sophisticated molecular addressing system during embryogenesis and then display molecules that provide the topologically precise, distinct targets for olfactory nerve growth cones.

The subventricular zone, a considerable distance posterior to the region where the olfactory bulb is formed, is the birthplace of neuronal precursor cells that are destined to form the olfactory bulb. Topological fate maps of this region reveal various specific positions of cells that are destined to generate distinct parts of the forebrain. A small region in the extreme anterior of the subventricular zone is the source of cells that will begin the migration to the region where the olfactory bulb is assembled (21,22; see FIG. 1). It was assumed that migratory cells are generated in an ordered fashion from these precursor cells and that the order of birth of daughter cells relates to their ultimate position in the topology of the olfactory bulb. As such cells are born they begin migrating along a narrow tube-like pathway bounded by glial cells but, unlike other regions of the embryonic brain, no radial glial processes are seen. The migrating spindle shaped cells remain in contact with neighboring cells in front, beside and behind and migrate as a stream only a few cells in diameter (21). Cell division continues while they migrate and maintain contacts. As cells in this stream reach the inner region of the developing olfactory bulb some form granule cells but many change directions and move outward toward their final positions near the surface of the bulb and become periglomerular cells. The dendrites of these cells become targets for the growth cones of olfactory cell axons that form synapses with them (22,23). A required consequence seems to be that this pattern of cell generation and migration relates directly to the specificity of the target receptor(s) that each cell will ultimately express. This process forms the precise and bilaterally symmetrical topological map of future targets for the growth cones extending from olfactory neurons born in the olfactory epithelium to the glomeruli in the olfactory bulb.

Serpentine receptors play a key and proven role as address molecules targeting the glomeruli. It seems important to examine various regions of the brain and embryo to determine where and when olfactory and VNO receptors are expressed. Clearly, it is reasonable to consider molecules expressed throughout the developing embryo.

There are many molecules other than the olfactory and VNO receptors that have been shown to play an important part in cell surface recognition (8). These molecules fulfill many of the addressing functions needed in an area code system by providing the equivalent of the country codes, area codes, regional codes, etc. One such example is O-CAM, one of a large number of cell surface receptors in the immunoglobulin supergene family (24,25). O-CAM is expressed on a subset of olfactory nerve axons that extend from the four zones of the olfactory epithelium to the specific zones of glomeruli in the olfactory bulb. This molecule is expressed on axons originating in three of the four zones of the olfactory epithelium and on one of the two zones from the VNO region. O-CAM thus seems to provide an excellent candidate for an area code molecule coding for geographic regions rather than for a specific cellular address. It is assumed that other, probably related receptors will be found on zones in which O-CAM is absent and that these will form part of the combinatorial code.

It may be possible to conceive of genetic, molecular and cellular mechanisms capable of accomplishing the assembly of the two thousand or so target sites in the olfactory bulb. As discussed above, neuronal precursor cells migrate considerable distances along stereotyped routes to lay out a precise, bilaterally symmetrical target map in the olfactory bulb. The mechanisms responsible are completely unknown. The only other example of this extraordinary level of migratory specificity is seen in the targeting of the axonal growth cones as they extend to form synapses in the olfactory bulb. In the absence of any good alternative, the possibility will be considered that members of the olfactory and VNO receptors are expressed in the cells that form the target arrays in the olfactory bulb. In this scenario, molecular interactions of these receptors with each other provide the required specificity for both migration and targeting. Cells may interact in such a way as to form the precise topological map of cells expressing target receptors. One intriguing possibility is suggested by the structure of the receptors themselves and by certain interesting patterns in which these structures are arrayed in the target maps. All of these receptors contain seven helical domains that traverse the membrane and arrange themselves so as to form a pocket at the cell surface. Studies have shown that these pockets provide specific sites for binding ligands. These receptors also display extra-cellular loops of varying size that provide additional specificity for interactions (26). Differences in the amino acid sequences within the domains forming the pockets and loops provide the individual specificity for ligand binding. There is speculation that this structure might also provide specificity for homophilic interactions (27).

Consider the notion that these combined binding sites provide the required specificity for both homophilic and heterophilic interactions of these receptors. Homophilic interactions could account for the target specificity known to occur as the olfactory axons seek specific glomeruli in the olfactory bulb. A possible method for the specificity of cell migration and bulb assembly derives from the observation that serpentine receptors with an unusual type of extracellular loop structure cluster together in both the olfactory epithelium and in the target bulb structure (28). Indeed, several studies suggest that glomeruli are arranged with receptors of similar structure displayed on adjacent glomeruli and within a specific region of the olfactory bulb (29). It seems possible that receptors differing only slightly in the amino acid sequence of the binding sites responsible for homophilic interactions could still interact with relatively high affinity. The binding constant difference could serve to guide neighbors to each other. Other adjacent cells could again have receptors with close but lower affinity. In this manner a type of affinity gradient could be established that, at least theoretically, could help explain the relationships maintained among cells as they migrate and assemble the target map in the olfactory bulb. Such a gradient of receptor affinities would also aid the growth cones of olfactory neurons as they seek their targets in the bulb.

The genetic programs are sophisticated enough to generate and maintain one thousand or more cells, each expressing one receptor gene. Elaborate genetic controls must function to maintain the expression of a single, specific serpentine receptor gene in each of the olfactory stem cells and in its daughter olfactory neurons as they continue to be born throughout life. Furthermore, these controls must allow the expression of only one of the two alleles present in each cell (30). The complexity of this genetic problem is very reminiscent of the similar situation seen in the immune system where sophisticated alterations are made in the germline DNA as specific B or T cells are generated. There too only a single allele is expressed in each cell. The altered DNA sequences are replicated for the life of a stem cell thus accounting for the lineage memory. Genetic switching therefore remains an attractive aspect of the Area Code Hypothesis, particularly for the control of the expression of the serpentine receptors discussed here. Indeed, it is extremely difficult to imagine that a mechanism utilizing only transcription factors et cetera is capable of mimicking the immune system's single-allele expression and stem cell-specific receptor expression.

Genetic Switches Known to Function in Various Organisms: The earliest proven example occurred of developmentally controlled genetic switching occurred in large colonies of Cyanobacter over two billion years ago (31,32). The same types of cyanobacteria exist today and form large colonies identical to those in the fossil record. In this organism, DNA rings are excised from the germline cell's DNA to form somatic cells that can fix nitrogen for the use of the entire colony. There is good reason to believe that this type of genetic switch evolved very early and has been selected for use in numerous subsequent species because of its efficacy as a means of programming the formation of different cell lineages.

Numerous types of repeats and transposable elements have also been shown to play a role in chromosomal programs, wherein germline DNA is altered as specific cell types are formed. Ciliates, for example, use transposes to excise specific transposon-like elements from germline DNA as a part of the mechanism used to form the somatic macronucleus from the germline micronucleus (33,34). Excision of specific transposable elements occurs in Drosophila as polytene chromosomes are formed from the germline. In another example, it is now known that the telomeres in Drosophila are maintained by two different transposable elements (35). Ribosomal DNA, like telomeres, must be controlled and maintained during development. These chromosomal regions contain numerous tandem copies of rDNA. In D. melanogaster specific transposable elements (different from those that maintain telomeres) are associated with rDNA (36). It seems very possible that they aid in the recombination control required for the maintenance and amplification of these chromosomal regions. Numerous other examples of DNA alterations during development of other organisms can be found in the literature.

The mechanism by which DNA is excised during the development of the immune system is very closely related to many of the examples mentioned above. Indeed, the RAG-1 transposase is evolutionarily related to the enzymes responsible for transposable element rearrangements found in essentially all eukaryotes and even bacterial switches such as the invertrons (37-40). Ten to twenty percent of the DNA of most multicellular organisms is made up of mobile DNA elements, hence large numbers of genes coding for members of the transposase/recombinase family are found in these genomes and according to our hypothesis, some may function in normal development.

The list of confirmed examples of programmed alterations in DNA is now so long that one is quite safe in stating that not all of the repeats and elements that make up a significant part of all chromosomes are “junk DNA.” It therefore seems reasonable to examine the possibility that some of the transposon-related elements may play a role in programming the expression of such genes as the serpentine receptors. Again, no other known mechanisms that do not involve alteration of DNA seem adequate to perform the extraordinarily complex programming of gene expression that is discussed here.

One obvious ramification of developmentally programmed DNA alteration is that cells from fully differentiated tissues could not be used to clone new individuals. And in fact this seems to be the case despite the two widely quoted examples of cloning from “differentiated” tissues. Neither the cloning of Dolly from the udder of a sheep (41), nor the cloning of an adult frog from larval frog intestines (42) was proven to have been accomplished from a differentiated cell type. The Dolly experiment has not been repeated and, even after thirty-six years, no successful repeat of Gurdon's result has been accomplished using confirmed differentiated cells from adult frogs (43). In each case above, the cloned individual was the very rare outcome of numerous experiments, and in both cases an embryonic germ cell could have been the cell actually selected for cloning. This is possible since the sheep which served as a donor for Dolly was pregnant, and since the larval frog intestine is a known site of germ cell migration during development. In contrast to the above reports, the successful use of nuclei derived from blastula cells in the nuclear transplantation experiments pioneered by Briggs and King in 1952 (44) has been reproduced many times and similar procedures have been used by numerous scientists in a variety of species throughout the past forty-six years. Nuclear transplantation from blastulas is compatible with the Area Code Hypothesis because DNA switching has not yet occurred at this stage of development and the cells are therefore totipotential. Thus, in another embodiment, the invention provides a method for obtaining such totipotential germ cells that may have migrated to various tissues (e.g., udder of cows, gonads/testis) and are maintained among the differentiated cells. Such cells are useful as starting material for nuclear transplantation in cloning experiments. In one embodiment, the invention provides a method for producing a specific cell lineage or organ type or an organism comprising obtaining a cell by the method of the invention as described herein. The cell(s) is treated under conditions and for a time sufficient to produce the lineage, organ or organism. For example, methods of producing organisms include nuclear transplantation.

Are repeats and transposon-related elements present in the sequences of the multigene families of serpentine receptors? FIG. 2 illustrates one of many examples of the DNA sequences of regions containing genes coding for serpentine receptors. Two serpentine receptors are coded by the DNA sequence illustrated. Note the pattern of elements near both upstream control regions. It was observed that all known sequences of DNA containing families of serpentine receptors contain sequences related to mobile elements in the non-coding regions. As such, careful consideration should be given to the possibility that repetitive elements, including some of those illustrated here, have a role in programming the expression of the very large families of seven-transmembrane receptor genes.

The data discussed above provide strong support for the notion that such receptors are indeed expressed in numerous tissues other than the olfactory regions. However, the data available at this time do not provide topological details of the expression of these molecules over time and space in the developing embryo. It is predicted that each receptor will be expressed in a speckled pattern throughout the embryo similar to the locations of the last four digits of phone numbers in geographic locations where they are used repeatedly in combination with other digits to code for different telephone sites. This type of pattern might easily be mistaken for an experimental artifact. A possible example of this may have already been published (14). Monoclonal antibodies developed to fractions of chick embryos correlating to the size of olfactory receptors were used to study expression in chick embryos. Close examination of the expression of olfactory receptors in chick embryos before, during and after notochord formation (see FIG. 6 in ref. 14) reveals numerous such specks not seen in the control. The notochord does indeed express an olfactory receptor but the speckled appearance of other parts of these sections was not noted by the authors. Obviously, more experiments are needed. As one example, the transgenic mice used by Mombaerts et. al. (10) would provide an excellent source of embryos for the study of the expression of olfactory receptors in tissues other than the adult olfactory system illustrated in their publication.

Do seven-transmembrane receptors interact with each other as is predicted by the above discussion? No study has been uncovered bearing directly on this aspect of the hypothesis, but such experiments are feasible. Several of the available excellent methods were used by Yoshihara et al. (24) in their studies of homophilic interactions of O-CAM. An additional method (45) was used. If it can be shown that no homophilic or heterophilic interactions can occur among these receptors other molecules would have to be found to explain the known facts. However, no reasonable alternative hypotheses can be offered.

Is there a gradient of closely related receptors on the topological map of glomeruli on the olfactory bulb? While several publications referenced above suggest that this may be true, more work needs to be done. Structural and functional studies of olfactory receptors expressed on neighboring glomeruli are needed to test this notion. Single-cell PCR techniques should facilitate testing of this “receptor gradient” hypothesis.

Is the control of the expression of the one thousand or so different serpentine receptors due in part to DNA switches? By now there are so many confirmed examples of the role of DNA alterations in somatic cells of diverse organisms that this part of the hypothesis should be given serious consideration. Several experimental approaches are now capable of providing data relevant to this subject. PCR methods can be used to compare specific stretches of DNA in germ line and somatic cells. DNA libraries from both cell types can also be used to detect specific differences. Protocols are readily available since studies of such differences in cells of the immune system have become commonplace in recent years. It is suggested that experiments be carried out to test the notion that the immune system is not alone in the use of mobile-element-related genetic switches in developmental controls of cell lineages.

The finding that serpentine receptors are expressed in a large number of different tissues has led us to suggest that they may play a central role in coding for cell positioning during embryogenesis. According to this hypothesis, these and other less-specific receptors are used in a combinatorial strategy that provides molecular codes to cell surfaces. Cells use these cell surface codes to guide their assembly of complex three-dimensional structures. The genetic control mechanisms required for the control of these codes are so sophisticated that it is suggested they utilize genetic switches related to mobile elements to aid in the control of the expression of codes on embryonic cells. Recombinases from the very large family encoded by mobile elements are candidates for a role in such DNA alterations. Rag-1, a member of this large recombinase family, plays a key role in the genetic events that use mobile element-related switches during the development of the immune system (37,38). A homeodomain that is also found on some of these recombinases (including Rag-1) raises more intriguing questions (39,40).

References

1. Hood, L., Huang, H. V. and Dreyer, W. J. (1977) J. Supramol. Struct. 7, 531-559.

2. Dreyer, W. J. and Bennett, J. C. (1965). The molecular basis of antibody formation: A paradox. Proc. Nat. Acad. Sci. USA 54, 864.

3. Dreyer, W. J. (1984) in The Impact of Protein Chemistry on the Biomedical Sciences, eds. Schechter, A. N., Dan, A. and Goldberger, R. F. (Academic Press, New York).

4. Dreyer, W. and Roman, J. M. (1984) in Advances in Experimental Medicine and Biology. Gene Expression and Cell-Cell Interactions in the Developing Nervous System, eds.Lauder, J. M. and Nelson, P., Vol. 181 (Plenum Press, New York), pp. 87-97.

5. Kayyem, J. F., Roman, J. M., Von Boxberg, Y., Schwarz, U. and Dreyer, W. J.(1992) Eur. J. Biochem. 208, 1-8.

6. Kayyem, J. F., Roman, J. M., de la Rosa, E. J., Schwarz, U. and Dreyer, W. J.(1992) J. Cell Biol. 118, 1259-1270.

7. Vielmetter, J., Kayyem, J. F., Roman, J. M. and Dreyer, W. J. (1994) J. Cell Biol. 127, 2009-2020.

8. Molecular bases of axonal growth and pathfinding (1997) in Cell˜Tissue Research, eds. Drescher, U., Klein, R., Sthrmer, C., Faissner, A. and Rathjen, F. G., Vol. 29 (Springer-Verlag, Berlin), pp. 187-470.

9. Mombaerts, P. (1996) Curr. Opin. Neurobiol. 6, 481-6.

10. Mombaerts, P., Wang, F., Dulac, C., Chao, S. K., Nemes, A., Mendelsohn, M., Edmondson, J. and Axel, R. (1996) Cell 87, 675-86.

11. Ressler, K. J., Sullivan, S. L. and Buck, L. B. (1994) Cell 79,1245-55.

12. Vassar, R., Nagi, J. and Axel, R. (1994) Cell 74, 309-318.

13. Drutel, G., Arrang, J. M., Diaz, J., Wisnewsky, C., Schwartz, K. and Schwartz, J. C. (1995) Receptor Channels 3, 33-40.

14. Nef, S. and Nef, P. (1997) Proc. Natl. Acad. Sci. USA 94, 4766-71.

15. Vanderhaeghen, P., Schurmans, S., Vassart, G. and Parmentier, M. (1997)Biochem. Biophys. Res. Commun. 237, 283-7.

16. Bargmann, C. I. (1997) Cell 90, 585-7.

17. Dulac, C. (1997) Neuron 19, 477-80.

18. Dulac, C. and Axel, R. (1995) Cell 83, 195-206.

19. Herrada, G. and Dulac, C. (1997) Cell 90, 763-73.

20. Matsunami, H. and Buck, L. B. (1997) Cell 90, 775-84.

21. Lois, C., Garcia-Verdugo, J. M. and Alvarez-Buylla, A. (1996) Science 271, 978-81.

22. Luskin, M. B. (1993) Neuron 11, 173-89.

23. Klenoff, J. R. and Greer, C. A. (1998) J. Comp. Neurol. 390, 256-267.

24. Yoshihara, Y., Kawasaki, M., Tamada, A., Fujita, H., Hayashi, H., Kagamiyama, H. and Mori, K. (1997) J. Neurosci. 17, 5830-42.

25. Yoshihara, Y. and Mori, K. (1997) Cell Tissue Res. 290, 457-463.

26. Shepherd, G. M, Singer, M. S. and Greer, C. A. (1996) The Neuroscientist 2, 262-271.

27. Singer, M. S., Shepherd, G. M. and Greer, C. A. (1995) Nature 337, 19-20.

28. Kubick, S., Strotmann, J., Andreini, I. and 8reer, H. (1997) J. Neurochem. 69, 465-75.

29. Friedrich, R. W. and Korsching, S. I. (1997) Neuron 18, 737-752.

30. Chess, A., Simon, I., Cedar, H. and Axel, R. (1994) Cell 78, 823-34.

31. Carrasco, C. D. and Golden, J. W. (1995) Microbiology 141, 2479-2487.

32. Haselkorn, R. (1992) Annul Rev. Genet. 26,113-130.

33. Williams, K., Doak, T. G. and Herrick, G. (1993) EMBO J. 12, 4593-4601.

34. Jacobs, M. E. and Klobutcher, L. A. (1996) J Euk. Microbiol. 43, 442-452.

35. Pardue, M. L., Danilevskaya, O. N., Traverse, K. L. and Lowenhaupt, T. K. (1997) Genetica 100, 73-84.

36. Eickbush, T. H., Burke, W. D., Eickbush, D. G. and Lathe, W. C., III (1997) Genetica 100,49-61.

37. Xu, W., Rould, M. A., Jun. S., Desplan, C. and Pabo, C. O. (1995) Cell 80, 639650.

38. Ramsden, D. A., van Gent, D. C. and Gellert, M. (1997) Curr. Opin. Immunol. 9, 114-120.

39. Spanopoulou, E., Zaitseva, F., Wang, F.-H., Santagata, S., Baltimore, D. and Panayotou, G. (1966) Cell 87, 263-276.

40. Pietrokovski, S. and Henikoff, S. (1997) Mol. Gen. Genet. 254, 689-695.

41. Wilmut, I., Schnieke, A. E., McWhir, J., Kind, A. G. and Campbell, K. H. S. (1997) Viable offspring derived from fetal and adult mammalian cells. Nature 385, 810-813.

42. Gurdon, J. B. (1962) Dev. Biol. 4, 256-273. 43. Cloning: Nuclear Transplantation in Amphibia (1978), ed. McKinnell, R.(U. Minnesota Press, Minneapolis).

44. Briggs, R. and King, T. J. (1952) Proc. Natl. Acad. Sci. USA 38, 455463.

45. Suter, D. M., Pollerberg, G. E., Buchstaller, A., Giger, R. J., Dreyer, W. J. and Sonderegger, P. (1995) J. Cell Biol. 131, 1067-1081. 46. Glusman, G., Clifton, S., Roe, B. and Lancet, D. (1996) Genomics 37, 147-60.

47. Ressler, K. J., Sullivan, S. L. and Buck, L. B. (1993) Cell 73, 597-609.

48. Scott, J. W., Shannon, D. E., Charpentier, J., Davis, L. M. and Kaplan, C. (1997) J. Neurophysiol. 77, 1950-62.

49. Strotmann, J., Konzelmann, S. and Breer, H. (1996) Cell Tissue Res. 284, 34754.

50. Juilfs, D. M., Fulle, H. J., Zhao, A. Z., Houslay, M. D., Garbers, D. L. and Beavo, J. A. (1997) Proc. Natl. Acad. Sci. USA 94, 3388-95

Although the invention has been described with reference to the above examples, it will be understood that modifications and variations are encompassed within the spirit and scope of the invention. Accordingly, the invention is limited only by the following claims.

What is claimed is:

1. A method for analyzing nucleic acid switch patterns or profiles in a cell or cell sample comprising: comparing the nucleic acid of the cell with nucleic acid from a corresponding germline cell or other cell, wherein a difference in the nucleic acid is indicative of a nucleic acid switch.

2. The method of claim 1 wherein the nucleic acid switch is present in extrachromosomal, cell-free or cell-associated nucleic acid.

3. The method of claim 1, wherein the cell is a stem cell.

4. The method of claim 3, wherein the cell is neuronal, epidermal, endodermal, mesodermal, hematopoietic, or non-germ cell stem cell.

5. The method of claim 1, wherein the cell is a cell of the immune system.

6. The method of claim 5, wherein the cell is a B-cell lineage cell.

7. The method of claim 5, wherein the cell is a T-cell lineage cell.

8. The method of claim 1, wherein the nucleic acid is DNA.

9. The method of claim 1, wherein the nucleic acid is RNA.

10. The method of claim 8, further comprising contacting the DNA with at least one additional marker that detects DNA associated with a specific cell type.

11. The method of claim 1, wherein the nucleic acid is detected by magnetic resonance imaging.

12. The method of claim 1, wherein the nucleic acid is detected using a binding agent.

13. The method of claim 12, wherein the binding agent is labeled with a detectable label.

14. The method of claim 13, wherein the detectable label is selected from the group consisting of enzymes, radioisotopes, fluorescent compounds, colloidal metals, chemiluminescent compounds, phosphorescent compounds, and bioluminescent compounds.

15. The method of claim 1, wherein the binding agent is immobilized on a solid support.

16. The method of claim 1, wherein the comparing is performed on a microarray.

17. The method of claim 1, wherein the nucleic acid is contacted with two or more binding agents.

18. The method of claim 1, wherein the cell-type is developmental or lineage-specific.

19. A method for identifying a differentiation stage-specific cell type in a cell sample, said method comprising comparing nucleic acid obtained from the cells with corresponding germline or undifferentiated cell nucleic acid, wherein the presence of at least one gene switch in the nucleic acid in the sample is indicative of a differentiated cell in the sample.

20. The method of claim 19, wherein the nucleic acid switch is detected in extrachromosomal, cell-free or cell-associated nucleic acid.

21. The method of claim 19, wherein the cell is a stem cell.

22. The method of claim 21, wherein the cell is neuronal, epidermal, endodermal, mesodermal, hematopoietic, or non-germ cell stem cell.

23. The method of claim 19, wherein the cell is a cell of the immune system.

24. The method of claim 23, wherein the cell is a B-cell lineage cell.

25. The method of claim 23, wherein the cell is a T-cell lineage cell.

26. The method of claim 19, wherein the nucleic acid is DNA.

27. The method of claim 19, wherein the nucleic acid is RNA.

28. The method of claim 26, further comprising detecting at least one additional marker that detects DNA associated with a specific cell type.

29. The method of claim 19, wherein the nucleic acid is detected using a binding agent.

30. The method of claim 29, wherein the binding agent is labeled with a detectable label.

31. The method of claim 19, wherein the nucleic acid is detected by magnetic resonance imaging.

32. The method of claim 30, wherein the detectable label is selected from the group consisting of enzymes, radioisotopes, fluorescent compounds, colloidal metals, chemiluminescent compounds, phosphorescent compounds, and bioluminescent compounds.

33. The method of claim 19, wherein the binding agent is immobilized on a solid support.

34. The method of claim 19, wherein the comparing is performed on a microarray.

35. The method of claim 19, wherein the nucleic acid is contacted with two or more binding agents.

36. A method for identifying a stem cell or a stage in the stem cell lineage in a sample, said method comprising:
contacting nucleic acid obtained from cells in the cell sample with at least one binding agent specific for a particular lineage switch such that the binding agent binds specifically to the region of nucleic acid affected by a gene switch; and
detecting binding of the agent to a region of nucleic acid affected by the switch, wherein a particular switch is indicative of a stem cell stage.

37. The method of claim 36 further comprising comparing the nucleic acid containing the region affected by the gene switch with corresponding germ line or undifferentiated cell nucleic acid to determine the developmental stage or lineage of the cell.

38. The method of claim 36 wherein the cell sample is blood or a blood component.

39. The method of claim 36, wherein the cell sample contains cells of neuronal cell lineage.

40. The method of claim 36, wherein the cell sample contains cells of muscle cell lineage.

41. The method of claim 36, wherein the cell sample contains cells of epidermal cell lineage.

42. The method of claim 36 wherein the nucleic acid switch is in extrachromosomal, cell-free or cell-associated nucleic acid.

43. The method of claim 36, wherein the cell is a stem cell.

44. The method of claim 43, wherein the cell is neuronal, epidermal, endodermal, mesodermal, hematopoietic, or non-germ cell stem cell.

45. The method of claim 36, wherein the cell is a cell of the immune system.

46. The method of claim 45, wherein the cell is a B-cell.

47. The method of claim 45, wherein the cell is a T-cell.

48. The method of claim 36, wherein the nucleic acid is DNA.

49. The method of claim 36, wherein the nucleic acid is RNA.

50. The method of claim 36, further comprising contacting the nucleic acid with at least one additional marker that detects nucleic acid associated with a specific cell type.

51. The method of claim 36, wherein the nucleic acid is detected using a binding agent.

52. The method of claim 51, wherein the binding agent is labeled with a detectable label.

53. The method of claim 52, wherein the detectable label is selected from the group consisting of enzymes, radioisotopes, fluorescent compounds, colloidal metals, chemiluminescent compounds, phosphorescent compounds, and bioluminescent compounds.

54. The method of claim 51, wherein the binding agent is immobilized on a solid support.

55. The method of claim 36, wherein the comparing is performed on a microarray.

56. The method of claim 36, wherein the nucleic acid is contacted with two or more binding agents.

57. The method of claim 36, wherein the nucleic acid is detected by magnetic resonance imaging.

58. A method for identifying a cell in a cell sample indicative of a disease state or disease process or predisposition thereto, the method comprising: contacting nucleic acid from a cell suspected of having a disease with at least one binding agent specific for a nucleic acid switch such that the binding agent binds specifically to the nucleic acid or to a region of the nucleic acid indicative of a switch, wherein the specific binding of the binding agent indicates the presence of a region of nucleic acid affected by a switch, and wherein the presence of the particular switch is associated with a disease state or a disease process or predisposition thereto in the cell.

59. The method of claim 58, wherein the nucleic acid switch is detected in extrachromosomal, cell-free or cell-associated nucleic acid.

60. The method of claim 58, wherein the cell is a stem cell.

61. The method of claim 60, wherein the cell is neuronal, epidermal, endodermal, mesodermal, hematopoietic, or non-germ cell stem cell.

62. The method of claim 58, wherein the cell is a cell of the immune system.

63. The method of claim 62, wherein the cell is a B-cell lineage cell.

64. The method of claim 62, wherein the cell is a T-cell lineage cell.

65. The method of claim 58, wherein the nucleic acid is DNA.

66. The method of claim 58, wherein the nucleic acid is RNA.

67. The method of claim 65, further comprising contacting the DNA with at least one additional marker that detects DNA associated with a specific cell type.

68. The method of claim 58, wherein the nucleic acid is detected using a binding agent.

69. The method of claim 68, wherein the binding agent is labeled with a detectable label.

70. The method of claim 69, wherein the detectable label is selected from the group consisting of enzymes, radioisotopes, fluorescent compounds, colloidal metals, chemiluminescent compounds, phosphorescent compounds, and bioluminescent compounds.

71. The method of claim 68, wherein the binding agent is a ligand.

72. The method of claim 68, wherein the binding agent is immobilized on a solid support.

73. The method of claim 58, wherein the comparing is performed on a microarray.

74. The method of claim 58, wherein the nucleic acid is contacted with two or more binding agents.

75. The method of claim 58, wherein the cell or cell sample is derived from a tumor.

76. The method of claim 58, wherein the cell or cell sample is derived from brain tissue.

77. The method of claim 58, wherein the cell or cell sample is derived from a biological fluid.

78. The method of claim 77, wherein the biological fluid is urine, sputum, saliva, blood, or cerebrospinal fluid.

79. The method of claim 58, wherein the cell or cell sample is derived from prostate tissue.

80. A method for diagnosing a subject having a disease characterized by the presence of a particular nucleic acid switch, the method comprising:
contacting test nucleic acid obtained from a sample of cells of the subject with at least one binding agent specific for a nucleic acid switch associated with a specific disease such that the binding agent detects a region of nucleic acid affected by the switch, wherein the binding of the agent indicates the presence of the specific disease in the subject.

81. The method of claim 80, wherein the disease is breast or prostate cancer.

82. A method for obtaining a composition substantially enriched in a specific cell type, the method comprising:
contacting a sample of cells with at least one binding agent specific for a mobile element-related polynucleotide indicative of a specific cell type such that the binding agent binds specifically to a cell or cells in the sample that express the polypeptide or binds to the polynucleotide; and
separating the cell or cells bound by the binding agent from the sample, thereby obtaining a composition substantially enriched in the specific cell type.

83. The method of claim 82, further comprising separating the cell or cells bound by the binding agent by selecting for at least one additional marker associated with the specific cell type.

84. The method of claim 82, wherein the additional marker is CD-34, Thy-1, rho, Cdw109, a protocadherins, a serpentine receptor, a cell adhesion molecule (CAM) or other cell surface marker.

85. A method for producing a specific cell lineage or organ type or an organism, the method comprising obtaining a stem cell within the cell lineage by cloning a cell identified by the method of claim 1 or 19 and treating the cell under conditions and for a time sufficient to produce the specific cell lineage, organ or organism.

86. The method of claim 85, wherein the cell is a stem cell.

87. The method of claim 85, wherein the cell is neuronal, epidermal, endodermal, mesodermal, hematopoietic, or non-germ cell stem cell.

88. The method of claim 85, wherein the cell is a cell of the immune system.

89. The method of claim 85, wherein the cell is a B-cell lineage cell.

90. The method of claim 85, wherein the cell is a T-cell lineage cell.

91. The method of claim 84, wherein the stem cell is in the muscle cell lineage.

92. A method of obtaining a composition substantially enriched in a specific cell type comprising:
contacting a sample of non-virally infected cells with at least one binding agent specific for an envelope cell surface marker such that the binding agent binds specifically to a cell or cells having the marker in the sample; and
separating the cell or cells bound by the binding agent from the sample, thereby obtaining a composition substantially enriched in a specific cell type.

93. The method according to claim 92, further comprising separating the cell or cells bound by the binding agent by selecting for at least one additional marker associated with a specific cell type.

94. The method according to claim 93, wherein the additional marker is selected from the group consisting of CD-34, Thy-1, rho, Cdw109, protocadherins, serpentine receptors and cell adhesion molecules (CAMs).

95. The method of claim 92, wherein the binding agent is selected from the group consisting of a ligand and an antibody.

96. The method of claim 95, wherein the antibody is monoclonal or polyclonal or derivative thereof.

97. The method of claim 92, wherein the binding agent is immobilized on a solid support.

98. The method of claim 92, further comprising analyzing the DNA of the cells.

99. The method of claim 92, wherein the analyzing is on a microchip.

100. The method of claim 92, wherein the at least one binding agent is multiplexed such that more than one binding agent is utilized simultaneously.
